(19) United States
(12) Patent Application Publication
(10) Pub. No.: US 2021/0308065 A1
(43) Pub. Date: Oct. 7, 2021

(54) REMOTE MODULATION OF BICONTINUOUS NANOSPHERES FOR CONTROLLED DELIVERY APPLICATIONS

(71) Applicant: Northwestern University, Evanston, IL (US)

(72) Inventors: Evan A. Scott, Northfield, IL (US); Sharan Bobbala, Evanston, IL (US); Sean D. Allen, Evanston, IL (US)

(21) Appl. No.: 17/301,512

(22) Filed: Apr. 5, 2021

Related U.S. Application Data
(60) Provisional application No. 63/004,775, filed on Apr. 3, 2020.

(51) Int. Cl. A61K 9/50; A61K 41/00; A61K 38/38; A61K 31/4745; A61K 31/20; A61K 9/51

(52) U.S. Cl. CPC A61K 9/5031; A61K 41/0071; A61K 9/51; A61K 31/4745; A61K 31/20; A61K 38/385

(57) ABSTRACT

Provided herein are bicontinuous nanosphere nanocarrier that allow for slow, sustained release or allow for targeted release of target molecules. Further embodiments of the present invention provide bicontinuous nanosphere nanocarrier comprising a photosensitizer and a suitable polymer. Also provided are methods for making and using the photosensitive nanocarrier.

### CROSS-REFERENCE TO RELATED APPLICATIONS

This application claims priority to U.S. Provisional Application No. 63/004,775, filed on Apr. 3, 2020, which is incorporated herein by reference in its entirety.

### STATEMENT REGARDING FEDERALLY SPONSORED RESEARCH

This invention was made with government support under grant CAREER 1453576 and CBET-1806007 awarded by the National Science Foundation, grant HL132390 awarded by the National Institutes of Health National Heart, Lung, and Blood Institute, and grant AI137932 awarded by the National Institutes of Health National Institute of Allergy and Infectious Disease. The government has certain rights in this invention.

### REFERENCE TO A SEQUENCE LISTING SUBMITTED VIA EFS-WEB

Not applicable

### BACKGROUND

A key rationale for specifying the nanostructure of drug delivery vehicles is to beneficially alter the pharmacodynamics and biodistribution of loaded cargo molecules.¹ By altering the size and morphology, the employed nanostructure may increase on-target delivery and reduce off-target uptake of their bioactive payloads.² Despite these well-documented benefits, nanostructure-mediated delivery still results in non-specific cellular uptake, particularly by cells of the mononuclear phagocyte system in organs such as the liver and spleen.³ This presents a particular challenge when potent cytotoxic cargoes are delivered. Stimuli-responsive nanostructures may decrease such non-specific cytotoxicity by providing spatiotemporal control over cargo release.⁴

One difficulty in using stimuli-responsive nanostructures is that they are often fabricated via the self-assembly of block copolymers, resulting in sensitive and easily ruptured vehicles. In particular, nanocarriers with aqueous lumens, which are required for facile encapsulation and transport of both hydrophilic and hydrophobic payloads simultaneously, are quickly disrupted under the harsh conditions of intracellular compartments.⁵ For example, despite their known enhanced stability relative to liposomes during in vivo transport, polymersomes are equally unlikely to survive intact within the lysosome for more than a few hours regardless of their chemistry. Due to the instability of nanostructures in cells, there also exists difficulty for delivering payload-containing nanostructures in vivo. Therefore, a need exists for nanostructures that are intracellularly stable so that a payload can be retained or slowly released in cells. Depending on the identity of the payload, these intracellularly stable nanostructures can be applied in areas such as cancer treatment and immunization, as well as other fields where long-term retention or slow release of therapeutic molecules in endosomal compartments is desired.

### SUMMARY OF THE INVENTION

The present invention provides intracellularly stable nanostructures that can be retained and slow release their contents into cells. The present invention also provides such nanocarriers for methods of treatment including cancer treatment and immunization, as well as treatments where long-term retention or slow release of therapeutic molecules in endosomal compartments is desired.

In another aspect, provided herein is an immunostimulatory composition comprising a bicontinuous nanosphere nanocarrier comprising poly(ethylene glycol)-block-poly(propylene sulphide) (PEG-b-PPS), mycolic acid (MA) and one or more target molecules, wherein the immunostimulatory composition is capable of stimulating an immune response to the target molecule. In some embodiments, the one or more target molecules is an antigen. In some embodiments, the one or more target molecules is an antigen.

In another aspect, the disclosure provides a bicontinuous nanosphere nanocarrier comprising poly(ethylene glycol)-block-poly(propylene sulphide) (PEG-b-PPS) and a photosensitizer. In some aspects, the nanocarrier further comprises mycolic acid. In some embodiments, the photosensitizer is hydrophobic and incorporates into an interior hydrophobic volume of the bicontinuous nanosphere. In some embodiments, the photosensitizer is pheophorbide A (PhA).

In some aspects, the one or more target molecules are hydrophobic. In some embodiments, the target molecule is hydrophilic. In some embodiments, the one or more target molecules is selected from the group consisting of a DNA molecule, an RNA molecule, a plasmid, a peptide, a protein, a small molecule, and combinations thereof. In some embodiments, the one or more target molecules is cytotoxic.

In some aspects, the PEG-b-PPS is PEG₁₇-b-PPS₇₅. In some embodiments, the PEG-b-PPS is benzyl functionalized.

In some aspects, the immunostimulatory composition additionally comprises a photosensitizer. In some embodiments, the photosensitizer is hydrophobic and incorporates into an interior hydrophobic volume of the bicontinuous nanosphere. In some embodiments, the photosensitizer is pheophorbide A (PhA).

In some aspects, the immunostimulatory composition is stable within a cell for at least 4 days, preferably at least 5 days.

In some aspects, the immunostimulatory composition additionally comprises a pharmaceutically acceptable carrier.

In another aspect, provided herein is a method of stimulating an immune cell, the method comprising contacting the immune cell with the immunostimulatory composition described herein, wherein the immunostimulatory composition stimulates the immune cell.

In some aspects, the immunostimulatory composition is taken up by the immune cell, and the composition is stable within the cell for at least 4-7 days.

In some aspects, the immunostimulatory composition slowly releases the one or more target molecules into the immune cell over at least seven days to stimulate the immune cell.

In further aspects, the immune cell is in vivo within a subject.

In a third aspect, provided herein is a method of stimulating an immune response to one or more target molecules in a subject in need thereof, the method comprising administering the immunostimulatory composition described herein in an amount effective to elicit an immune response.

In some aspects, the immune response comprises activation of one or more T cells in the subject. In some embodiments, the T cells are CD1b-restricted.

In another aspect, provided herein is a method for providing a photodynamic therapy to a subject comprising: (i) administering to the subject the immunostimulatory composition described herein comprising a photosensitizer; (ii) irradiating the composition comprising the nanocarrier for a time and under conditions sufficient to generate reactive oxygen species and release the one or more target molecules from the nanocarrier. In some embodiments, the nanocarrier is irradiated with light at an intensity between 5 and 200 mW/cm² for about 0.5 minutes to about 5 minutes. In some embodiments, the light has a wavelength of 385 nm to 740 nm. In some embodiments, the one or more target molecules is cytotoxic.

### BRIEF DESCRIPTION OF DRAWINGS

The patent or patent application file contains at least one drawing in color. Copies of this patent or patent application publication with color drawings will be provided by the Office upon request and payment of the necessary fee.

FIGS. 1A-1D show subcellular localization and retention of free or encapsulated FITC-BSA. (FIG. 1A) Representative confocal micrographs of RAW 264.7 cells immediately after a 4-h incubation with FITC-BSA or 48 h after a 4-h incubation with FITC-BSA. (FIG. 1B) Representative confocal micrographs of RAW 264.7 cells after 48 or 168 h after a 4-h incubation with BCNs loaded with FITC-BSA. All cells were stained with NucBlue for nuclei and LysoTracker Red for lysosomes, magnification 40× objective lens. (FIG. 1C) Integrated density of fluorescent signal for randomly selected cells that underwent no treatment or treatment with either free FITC-BSA or FITC-BSA BCNs. Data was obtained for 30 cells per condition and 3 separate experiments using ImageJ Software. Cells were analyzed immediately after a 4 h incubation with treatment or 44 h after a 4 h incubation with treatment (48 h total). Statistical analysis was performed using 2-way ANOVA, *** p<0.001 and **** p<0.0001. (FIG. 1D) Flow cytometric analysis of BODIPY fluorescence in RAW 264.7 cells at various timepoints after a 30-minute BODIPY-BSA BCN incubation (n=4; error bars=s.d.).

FIGS. 2A-2F show physical characterization of PhA-BCNs. (FIG. 2A) TEM micrograph of PhA-BCN. Scale bar=100 nm. (FIG. 2B) SAXS curve of PhA-BCN, with labelled Bragg peaks. (FIG. 2C) Encapsulation efficiency of PhA and hydrophilic cargoes calcein and FITC-BSA (n=3, error bars=s.d.). (FIG. 2D) Percentage of fluorescence signal in the supernatant after irradiation and centrifugation of BCNs loaded with PhA or ethyl eosin, n=4, error bars=s.d. (FIG. 2E) Generation of singlet oxygen assayed by percent decrease in DPBF absorbance for free solubilized PhA, blank BCNs, and PhA-BCNs (n=4, error bars=s.d.). (FIG. 2F) MTT assay of cell viability with or without irradiation for cells treated with free PhA or PhA-BCNs, 12 h after irradiation. For all subfigures * p<0.0001.

FIGS. 3A-3G show morphological transition of BCNs into micelles by photo-oxidation. (FIG. 3A) Diagram of PhA-BCNs degrading into micelles after irradiation. (FIG. 3B) Schematic of BCN isolation from micelles by centrifugation at 10,000 rcf. (FIG. 3C) TEM micrograph of micelle nanostructures isolated from the supernatant of centrifuged samples after irradiation. (FIG. 3D) TEM micrograph of BCNs isolated from the pellet of centrifuged samples after irradiation. Scale bar of TEM micrographs=200 nm. (FIG. 3E) DLS size distributions of samples shown in (FIG. 3C) and (FIG. 3D) (n=3, error bars=s.d.). (FIG. 3F) SAXS curve of the supernatant shown in (FIG. 3C), shown with the SAXS curve of non-irradiated PhA-BCNs (same curve as shown in FIG. 2B). Supernatant SAXS data was fitted with a micelle model fit using SASView. SAXS data was arbitrarily offset between BCN and supernatant data. (FIG. 3G) Confocal images of PhA-BCNs loaded with FITC-BSA, internalized by RAW 264.7 cells, with or without irradiation, magnification 63× objective lens.

FIGS. 4A-4C show cytosolic delivery of cytotoxic cargo. (FIG. 4A) MTT viability assay of RAW 264.7 cells. Cells were treated with free camptothecin (CT) or PhA-BCNs loaded with CT at three concentrations (0.63, 1.56, and 3.1 μg/mL) for 8 h before a group was irradiated. Cells were irradiated for 2.5 minutes and incubated further to allow for the induction of cell death, either 24 or 48 h total. Statistical evaluation of the treatment groups at the (FIG. 4B) 1.56 μg/mL and (FIG. 4C) 0.63 μg/mL concentrations. PhA BCN −Light and +Light data at 24 and 48 h are duplicated between (FIG. 4B) and (FIG. 4C), displayed as such for ease of comparison. For all subfigures n=4, error bars are s.d, statistical analysis by 2-way ANOVA.

FIGS. 5A-5C show formation of PhA-loaded BCNs (PhA-BCNs) from oxidation-sensitive PEG-b-PPS polymer. (FIG. 5A) PEG-b-PPS polymer structure. Sulfide oxidation into sulfoxide and sulfone groups increases hydrophilicity. (FIG. 5B) Pheophorbide A structure. (FIG. 5C) BCN self-assembly and cargo loading were performed using flash nanoprecipitation (FNP).

FIG. 6 shows a representative confocal image of RAW 264.7 cells 44 H after a 4 H of incubation with FITC-BSA BCNs and subsequent washing. All cells were stained with NucBlue for nuclei and LysoTracker Red for lysosomes. Scale bar=40 μm.

FIG. 7 shows low magnification negative-stain TEM micrograph BCNs. Scale bar=500 nm.

FIG. 8 shows change in turbidity of BCN solution after irradiation. A comparison between loaded PhA and ethyl eosin (EE) in their capacity to induce an increase in transmittance compared to non-irradiated controls. Also included are photographs of the solutions before and after irradiation (left to right). n=3, error bars=s.d., *** p<0.001.

FIG. 9 shows Generation of singlet oxygen assayed by percent decrease in DPBF absorbance for free solubilized PhA, blank BCNs, and PhA BCNs. n=4, error bars=s.d. Irradiated at 50 mW/cm².

FIGS. 10A-10B show Photo-oxidation induced release of hydrophilic cargo. (FIG. 10A) Release of calcein, FITC-BSA, and PhA from BCNs after photo-irradiation for varying times (n=4, error bars=s.d.) compared to non-irradiated samples. (FIG. 10B) Fluorescence loss represents the percent decrease in the fluorescence compared to non-irradiated samples. n=4, error bars=s.d.

FIG. 11 shows ROS generation upon irradiation of cells. Intracellular ROS generation measured by increase in fluorescence of DCFDA. RAW 264.7 cells were left untreated or were treated with free PhA or PhA BCNs and were irradiated for 2.5 minutes.

FIG. 12 shows low magnification negative-stain TEM micrograph of irradiated supernatant. Scale bar=1 μm.

FIG. 13 shows distribution of micelle diameters measured from TEM micrographs of irradiated supernatant. n=100 measurements, three separate micrographs.

FIG. 14 shows confocal images of Pheophorbide A-BCNs loaded with FITC-BSA, internalized by RAW 264.7 cells, with or without irradiation. All cells were stained with Lysotracker Blue for lysosomes. White arrows in the top row represent colocalization signal of lysotracker with FITC-BSA and Pheophorbide A. Scale bar is 25 μm.

FIG. 15 shows that BCNs have higher mycolic acid (MA) encapsulation efficiency than poly(lactic-co-glycolic acid) (PLGA) NP. Encapsulation efficiency of MA in BCN is 95+/−3% and 43+/−2% in PLGA NP.

FIG. 16 shows that loading of MA does not modulate BCN structure. A cryogenic transmission electron microscopy image of a BCN loaded with MA.

FIGS. 17A-C show vaccination with MA-loaded BCN allows for higher activation and proliferation compared to unloaded BCN and MA-loaded or unloaded PLGA. A) Percentage of CD44+ DN1 T cells or B) percentage of proliferating DN1 T cells (CD1b-restricted T cells from humanized mice) taken from draining lymph nodes (DLNs) and lungs of hCD1Tg mice 7 days post 3e6 intravenous DN1 T cell adoptive transfer and 6 days after intratracheal vaccination of Blank BCN, Blank PLGA NP, MA BCN, and MA PLGA. C) DN1 T cell proliferation of hCD1Tg DLN, 7 days post 3e6 IV DN1 T cell adoptive transfer and 6 days after intratracheal vaccination as shown with cell trace BV541. * p<0.01, ** p<0.001

FIGS. 18A-B show that adoptively transferred DN1 T cells are activated and proliferate 6 and 3 weeks after MA BCN vaccination in hCD1Tg mice (humanized transgenic mice that respond to lipid antigens similar to humans). A) Cell trace of CD44+ DN1 T cells after 3 or 6 weeks. B) Percentage of CD44+ DN1 T cells in DLN, lung, or spleen 3 or 6 weeks after intratracheal vaccination of MA BCN. N=2 or 3.

FIGS. 19A-B demonstrate that MA BCN and MA pulsed BMDC vaccination shows differences in activation and proliferation of DN1 T cells 6 weeks, but not 1 week after vaccination. A) Percentage of CD44+ DN1 T cells after vaccination to show activation. B) Percentage of cell trace-DN1 T cells after vaccination to show proliferation. N=2 or 3, ** p<0.01.

### INCORPORATION BY REFERENCE

All publications, patents, and patent applications mentioned in this specification are herein incorporated by reference to the same extent as if each individual publication, patent, and patent application was specifically and individually indicated to be incorporated by reference.

### DETAILED DESCRIPTION OF THE INVENTION

The present disclosure provides intracellularly stable nanostructures that can be retained and slow release their contents into cells and photosenstivie nanostructures which can be triggered to release their contents when exposed to a light source. The present invention also provides such nanocarriers for methods of treatment including cancer treatment and immunization, as well as treatments where long-term retention or slow release of therapeutic molecules in endosomal compartments is desired.

The present disclosure describes photosensitive bicontinuous nanosphere nanocarrier. As demonstrated herein, PEG-b-PPS bicontinuous nanosphere nanocarriers comprising a photosensitizer allow for controllable release of therapeutic target agents from the nanocarrier. These carriers can maintain cytotoxic payload within a cell while extending cell viability. Methods for making and using these photosensitive nanocarriers are also provided.

In another embodiment, the present disclosure describes an immunostimulatory composition comprising a stable bicontinuous nanosphere nanocarrier described herein. The stable bicontinuous nanosphere nanocarrier comprises or consists of PEG-b-PPS, mycolic acid (MA), and one or more target molecules, which stimulates an immune response to the target molecule. These bicontinuous nanospheres can have extended release of their target molecules, allowing for increased viability of the cells and for slow release or the target molecules (including therapeutic molecules) over a number of days. These immunostimulatory compositions can stimulate an immune cell. The BCN carriers can maintain cytotoxic payload within a cell while extending cell viability. Methods for making the bicontinuous nanosphere nanocarrier and using the immunostimulatory composition are also provided.

Nanocarriers described herein are characterized by complex or vesicular nanoarchitectures capable of encapsulating or comprising as part of the nanocarrier a target molecule. The nanoarchitectures of the nanocarriers may be bicontinuous and may be characterized as, for example, nanospheres, filomicelles, cubisomes, vesicles, tubules, nested vesicles, filiments, and vesicular, multilamellar and tubular polymersomes. In some embodiments, the nanocarrier is a bicontinuous nanosphere. Specifically, the bicontinuous nanosphere is capable of slow release of the target molecule it is carrying. The addition of a photosensitizer molecule can also then allow for the triggered release of the target molecule, if desired.

As used herein, “bicontinuous nanosphere (BCN)” refers to a morphology characterized by a continuous internal phase composed of two immiscible continuous phases; (i) an internal aqueous lattice (e.g., internal cubic (e.g. Im3m) lattice of aqueous channels, other internal cubic organizations, including diamond (Pn3m) and dyroid (Ia3d)) that traverse (ii) an extensive hydrophobic interior volume. Based on small angle X-ray scattering (SAXS) analysis, BCN have primitive type cubic internal organization (Im3m) as confirmed by Bragg peaks with relative spacing ratios at √2, √4, and √6. BCNs are the polymeric equivalent of lipid cubosomes and are lyotropic. BCN can incorporate both hydrophobic and hydrophilic payload molecules.

The BCN nanocarriers described herein further comprises mycolic acid (MA) and one or more targeting molecules. The nanocarriers were surprisingly found to have intracellularly stable nanostructures that can be retained inside a cell without toxicity and allow for the slow release of the target molecule carried. These nanocarries can be used in immunostimulatory compositions to elicit an immune response to the target molecules. These nanocarriers can also be used for [symbol] This allows for the slow and sustained release of the target molecule.

In some aspects, the nanocarrier include a photosensitizer molecule. These nanocarriers carrying the photosensitizer molecule and the target molecule can be irradiated and allow for the quick release of the target molecule in a cell.

As used herein, “photosensitizer” and “photosensitizer molecule” are used interchangeably and refer to a photo-oxidizer or photothermal molecule that, upon exposure to light, produce singlet oxygen, reactive oxygen species, or combinations thereof causing oxidation of surrounding polymers, compounds, cells, tissues, and biomolecules. Suitable photosensitizers are known in the art. Photosensitizers may include, but are not limited to, pheophorbide A (PhA), methylene blue, toluidine blue, protoporphyrin, ethyl eosin, pthalocyanine, indocyanine green, bacteriochlorin, phthalocyanines, chlorins, bodipy derivatives and porphyrin derivatives like benzoporphyrins. See, for example, Lan et al. (“Photosensitizers for photodynamic therapy,” Advanced Healthcare Materials, 2019, 8 (13)), which is incorporated herein by reference in its entirety.

Photosensitizers are irradiated under conditions and for a time suitable to produce singlet oxygen, reactive oxygen species, or combinations thereof. In some embodiments, the light used for irradiation has a wavelength of 20 to 900 nm. In some embodiments, the light has a wavelength of 385 to 740 nm. In some embodiments, the light used for irradiation has an intensity between about 5 mW/cm² and about 200 mW/cm² (e.g., 5 mW/cm², 10 mW/cm², 15 mW/cm², 20 mW/cm², 25 mW/cm², 30 mW/cm², 35 mW/cm², 40 mW/cm², 45 mW/cm², 50 mW/cm², 55 mW/cm², 60 mW/cm², 65 mW/cm², 70 mW/cm², 75 mW/cm², 80 mW/cm², 85 mW/cm², 90 mW/cm², 95 mW/cm², 100 mW/cm², 110 mW/cm², 120 mW/cm², 130 mW/cm², 140 mW/cm², 150 mW/cm², 160 mW/cm², 170 mW/cm², 180 mW/cm², 190 mW/cm², or 200 mW/cm²). In some embodiments, the photosensitizer is irradiated for at least 30 second, at least 1 minute, at least 2 minutes, at least 3 minutes, at least 4 minutes, at least 5 minutes, at least 6 minutes, at least 7 minutes, at least 8 minutes, at least 9 minutes, at least 10 minutes, at least 15 minutes, at least 20 minutes, or at least 30 minutes. See, for example, Abrahamse et al. (“New photosensitizers for photodynamic therapy,” Biochemical Journal, 473 (4):347-364) and Lan et al. (“Photosensitizers for photodynamic therapy,” Advanced Healthcare Materials, 2019, 8 (13)), each of which are incorporated herein by reference in their entirety and which describe photosensitizers and suitable conditions and methods for use thereof.

Production of singlet oxygen or reactive oxygen species (ROS) can be measured by any suitable means known in the art. In some embodiments, singlet oxygen or ROS production is measured using chemical oxidation of 1,3-diphenylisobenzofuran (DPBF). Using DPBF, production of oxidizing species can be calculated as a percentage reduction in DPBF absorbance at 415 nm following exposure to the oxidizing species as compared to initial DPBF absorbance.

In some embodiments, the nanocarriers are made using flash-nanoprecipitation. As used herein, “flash nanoprecipitation” (FNP) refers to a process in which a block copolymer is assembled into a nanocarrier architecture. FNP may also be used to load the nanocarrier with a photosensitizer, a target molecule, or combinations thereof as described herein. FNP methods employ multi-stream mixers in which an organic solution and a block copolymer dissolved in a suitable solvent are impinged upon an aqueous solution under turbulent conditions and subsequently introduced into an aqueous reservoir. The supersaturated conditions generated by the turbulent mixing induces precipitation of the block copolymer for stabilization of monodisperse nanoparticles, which may be loaded with a hydrophilic molecule, a hydrophobic molecule, a photosensitizer, or combinations thereof as described herein. Mixing occurs over millisecond timescales and is followed by transfer to a reservoir comprising a second aqueous solution to strip away solvent still associating with the aggregated block copolymer. Flash nanoprecipitation advantageously allows for loading hydrophilic target molecules as well as hydrophobic target molecules, such as, but not limited to, the photosensitizers described herein. Methods for flash nanoprecipitation are described in US 2018/0022878, which is incorporated herein by reference in its entirety.

The nanocarriers may be formed from suitable amphiphilic copolymers. Amphiphilic copolymers are comprised of sub-units or monomers that have different hydrophilic and hydrophobic characteristics. Typically, these sub-units are present in groups of at least two, comprising a block of a given character, such as a hydrophobic or hydrophilic block. Depending on the method of synthesis, these blocks could be of all the same monomer or contain different monomer units dispersed throughout the block, but still yielding blocks of the copolymer with substantially hydrophilic and hydrophobic portions. These blocks can be arranged into a series of two blocks (diblock) or three blocks (triblock), or more, forming the backbone of a block copolymer. In addition, the polymer chain may have chemical moieties covalently attached or grafted to the backbone. Such polymers are graft polymers. Block units making up the copolymer can occur in regular intervals or they can occur randomly making a random copolymer. In addition, grafted side chains can occur at regular intervals along the polymer backbone or randomly making a randomly grafted copolymer. The ratio of the hydrophobic to hydrophilic blocks of the copolymer will be selected such that the soluble and insoluble components are balanced and suitable aggregation for the desired architectures.

Suitable amphiphilic copolymers of the present invention are those polymers with a low glass transition temperature (Tg) hydrophobic block, typically below 0° C. or between about −70° C. and about 0° C. (i.e., less than about 10° C., 0° C., −5° C., −10° C., −20° C., −25° C., −30° C., −40° C., −45° C., −50° C., −60° C. or −70° C. and greater than about −70° C., −60° C., −50° C., −45° C., −40° C., −30° C., −25° C., −20° C., −10° C., or −5° C.). Polymers within this range will exhibit high mobility between polymer chains. Polymers which fit these characteristics include, without limitation, poly(ethylene glycol) (PEG), poly(propylene sulfide) (PPS), poly(ethylene sulfide), polycaprolactone, poly(dimethylsiloxane) and polyethylene. Polymers may also include chemical modifications or end caps. Chemical modification and end caps may include, but are not limited to, thiol, benzyl, pyridyl disulfide, phthalimide, vinyl sulfone, aldehyde, acrylate, maleimide, and n-hydroxysuccinimide groups. The chemical modification of the polymer may add a charged residue to the polymer or may be used to otherwise functionalize the polymer.

In some embodiments of the present invention, the polymer is poly(ethylene glycol)-block-poly(propylene sulfide) (PEG-b/-PPS). In one embodiment, the polymer is PEG₁₇-bl-PPS₃₀. Advantages of the PEG-b-PPS nanocarrier system include rapid gram-scale fabrication, stability for months to years when loaded with a photosensitizer, high loading efficiency for proteins (e.g., ˜70% for albumin) and small molecules (e.g, >90% for imiquimod derivatives), redox-sensitivity for intracellular delivery, amenability to multimodal imaging, and controllable release when combining the photosensitizer with another molecular payloads.

As used herein, the term “organic phase solution” refers collectively to the solution comprising the process solvent, the amphiphilic copolymer, and optionally one or more target molecules. The process solvent may be any water miscible organic solvent in which the hydrophobic block of the amphiphilic copolymer is soluble. The proper process solvent will be selected based on the identity and characteristics of the amphiphilic copolymer selected. Water miscible organic solvents are known in the art and include, without limitation, tetrahydrofuran (THF), dimethylformamide (DMF), dimethyl sulfoxide (DMSO), acetonitrile, methanol, 1,2-Butanediol, 1,3-Butanediol, 1,3-Propanediol, 1,4-Butanediol, 1,4-Dioxane, 1,5-Pentanediol, 1-Propanol, 2-Butoxyethanol, 2-Propanol, acetaldehyde, acetic acid, acetone, butyric acid, diethanolamine, diethylenetriamine, dimethoxyethane, dimethyl sulfoxide, dimethylformamide, ethanol, ethylamine, ethylene glycol, formic acid, furfuryl alcohol, glycerol, methyl diethanolamine, methyl isocyanide, propanoic acid, propylene glycol, pyridine, tetrahydrofuran, triethylene, and glycol. The organic phase solution may optionally comprise one or more lipophilic target molecules, one or more hydrophobic target molecules, a photosensitizer, or combinations thereof. In one embodiment, the process solvent is THF. In one embodiment, the process solvent is DMSO. In one embodiment, the process solvent is DMF.

As used herein, the term “aqueous phase solution” refers collectively to the solution comprising an aqueous nonsolvent and optionally one or more target molecules. The aqueous solution can comprise an aqueous nonsolvent solution comprising pure water, a buffering agent, salt, colloid dispersant or inert molecule, or combinations thereof. The aqueous phase solution may comprise one or more buffers, one or more salts, and one or more supplemental additive agents, such as inert diluents, solubilizing agents, emulsifiers, suspending agents, adjuvants, wetting agents, reducing agents, isotonic agents, colloidal dispersants and surfactants. In some embodiments, the aqueous nonsolvent is phosphate-buffered saline (PBS). In some embodiments, the salt is a kosmotropic salt. In some embodiments, the buffer is selected form common buffers used for biochemical reactions and cell culture, including phosphate buffer saline (PBS), (4-(2-hydroxyethyl)-1-piperazineethanesulfonic acid) (HEPES), tris(hydroxymethyl)aminomethane (Tris), citric acid, and 3-(N-morpholino)propanesulfonic acid (MOPS). In some embodiments, the salt is a kosmotropic salt. In some embodiments, the salt is selected from the group consisting of, sodium chloride, ammonium acetate, potassium chloride, monopotassium phosphate, disodium phosphate, sodium acetate, and zinc chloride.

The aqueous phase solution is formulated in a manner sufficient to maintain the stability of any target agent suspended or dissolved therein. For example, it is envisioned that if the additive target agent is selected from the group consisting of a DNA molecule, an RNA molecule, and a protein molecule, the aqueous solution will have a proper pH and salinity such that the target molecule will maintain proper folding and stability while in solution. In some embodiments, the aqueous phase solution will have a physiologically relevant pH and salinity appropriate for loading biological macromolecules into the nanocarriers. In one embodiment, the aqueous phase solution comprises between about 0 mM and 200 mM salt. In one embodiment the aqueous phase solution comprises less than or equal to 150 mM salt. In one embodiment, the aqueous phase solution has a pH between about 2.0 and 12.0. In one embodiment the aqueous phase solution has a pH between about 5.0 and 9.0. In one embodiment the aqueous phase solution has a pH between about 7.0 and 8.0.

As used herein, the term “aqueous nonsolvent” refers to the water or other aqueous solvent solution present in the aqueous phase solution or in the reservoir solution. The amphiphilic copolymer is not solvent in the nonsolvent, and the nonsolvent acts to strip the water miscible organic solvent away from the amphiphilic copolymer during the process of flash nanoprecipitation.

In another aspect of the invention, the immunostimulatory composition comprising the bicontinuous nanosphere nanocarriers are made and include mycolic acid (MA) and one or more target molecules. The one or more target molecules may be added to the organic phase solution, the aqueous phase solution, the reservoir, or combinations thereof. In some embodiment, a target molecule is included with the amphiphilic copolymer in the organic phase solution. In some embodiments, the target molecule is present in the aqueous phase solution. In some embodiments, a first target molecule is included in the organic phase solution and a second target molecule is included in the aqueous phase solution. In some embodiments, the target molecule is included in the reservoir. The target molecule is combined with the amphiphilic copolymer in a ratio of 1:4 to 10:1 by weight or charge. In one embodiment, the target molecule is mixed with the amphiphilic copolymer in at least a 1:2 ratio by weight. Preferably the target molecule is present in the mixture after mixing at a concentration of at least 0.1% by weight, but more preferably the concentration of target molecule is at least 0.2% by weight. In some embodiments, the target molecule is included at between 0.1% and 20% by weight, between 1% and 15% by weight or between 1.5% and 12% by weight. The temperature and the pressure of the organic phase solution, the aqueous phase solution or the mixture thereof can be altered to allow complete dissolution of both the amphiphilic copolymer and the target molecule while maintaining a liquid phase.

As used herein, the term “target molecules” or “target agent” refers to any molecule to be loaded into the nanocarriers according to embodiments of the present invention. The target molecule may be hydrophobic, hydrophilic, lipophilic or amphiphilic. The target molecule may include hydrophilic macromolecules such as RNA, DNA, plasmids, peptides, antibodies, proteins, fluorophores, carbohydrates, small molecule drugs, water soluble synthetic polymers and combinations thereof. Target molecules also include adhesive or targeting moieties such as cell specific antibodies which target the nanocarrier to a specific cell type or target of interest. Examples of other target molecules that may be added to nanoparticles by this process can be selected from, but are not limited to, the known classes of drugs including immunosuppressive agents such as cyclosporins (cyclosporin A), immunoactive agents, analgesics, anti-inflammatory agents, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiac inotropic agents, contrast media, corticosteroids, cough suppressants (expectorants and mucolytics), diagnostic agents, diagnostic imaging agents, diuretics, dopaminergics (antiparkinsonian agents), haemostatics, immunological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasodilators, xanthines, anti-oxidants, preservatives, vitamins, nutrients, adjuvants, antigents, MRI contrast agents, metal (i.e., gold, iron oxide, and the like), nanomaterials (i.e., quantum dots, micelles), temperature sensitive polymers (i.e., Poly(N-isopropylacrylamide)), polymer-drug conjugates, and biologics (referring collectively to any carbohydrate, protein, polypeptide, nucleic acid, combinations thereof and the like). Target molecules may also include combinations of, complexes of, mixtures of or other associations of any of the target molecules listed.

In some embodiments, the target molecule is cytotoxic or is employed at a cytotoxic dose. As used herein, “cytotoxic” refers to a molecule or conditions that promotes cell death such as by apoptosis, necrosis, or autophagy. Suitable cytotoxic agents are known and described in the art. Suitable cytotoxic agents include, but are not limited to, chemotherapeutics such as camptothecin, cisplatin, doxorubicin, and methotrexate. Also, see, for example, the list by McDonnell, assembled June 2016 and available on the World Wide Web at the cancernetwork.com under the title “Chemotherapeutic Agents and Their Uses, Dosages, and Toxicities.” In some embodiments, the cytotoxic agent is a chemotherapeutic. In some embodiments, the cytotoxic agent is the photosensitizer molecule.

In some embodiments, the target molecule is a pharmaceutical target or a pharmaceutical drug. Suitable pharmaceutical targets or pharmaceutical drugs would be known and understood to one skilled in the art.

In some embodiments, the target molecule is an antigen. As used herein, the term “antigen” refers to any molecule that is recognized by the immune system and that can stimulate an immune response. In some embodiments, the antigen is a peptide or protein or a lipid component. In another embodiment, the antigen is a component of an infectious agent. As used herein, “lipid antigen” refers to a lipid moiety present on the exterior surface of or within an infectious agent and that elicits an immune response in a subject. Suitable lipid antigens may be lipid components of the cell walls or cell membranes of infectious agents. The range of known self and foreign lipid antigens that are presented by CD1 molecules includes extremely diverse types of lipids including lipopeptides, diacylglycerolipids, sphingolipids, mycolates, phosphomycoketides, but also small molecules. Among these are self-lipids, such as sulfatide or isoglobotrihexosylceramide (iGb3), but also many microbial antigens from pathogenic bacteria, such as didehydroxymycobactin or glucose monomycolate. Suitable lipid antigens are known and described in the art and may include, but are not limited to, mycolic acid, dieoxymycobactin, mannosyl phosphomycoketide, Mtb total lipid extract (Tlip), sulfoglycolipid (SGL), phosphatidyl mannoside 2 (PIM2), phosphotidyl mannoside 6 (PIM6), lipoarabinomannan (LAM), trehalose dimycolate (TDM), glucose monomycolate (GMM), Didehydroxymycobactin (DDM-838), Glucose Monomycolate (GMM), Mannosyl-1β-phosphomycoketide (β-MPM), and Phosphatidylinositol mannoside-4 (PIM-4), α-galacturonosyl ceramide (GalA-Gsl), diacylglycerol glycolipids from the pathogenic bacterium Borrelia burgdorferi (BbGl-2c), phenyl 2,2,4,6,7-pentamethyldihydrobenzofuran-5-sulfonate (PPBF), α-galactosylceramide (αGalCer), palmitic acid, isoglobotrihexosylceramine, sulfatide, phosphatidylcholine, spingosine and variants thereof, fatty acid variants, and combinations thereof. See, for example, Schiefner et al. (“Presentation of lipid antigens by CD1 glycoproteins,” Curr Pharm Des., 2009, 15 (28):3311-3317) and Zajonc (“The CD1 family: serving lipid antigens to T cells since the Mesozoic era,” Immunogenetics, 2016, 68 (8):561-576), each of which is incorporated herein by reference. In some embodiments, the lipid antigen is a total lipid extract from a bacterium, fungi, or other infectious agent. In some embodiments, the lipid antigen is a lipid specific to a bacterium, fungi, or other infections agent.

As used herein, “peptide antigen” and “protein antigen” are used interchangeably and refer to peptide moieties specific to an infectious agent that elicit an immune response in a subject. Suitable protein antigens may be a peptide component from an infectious agent. Suitable Mycobacterium tuberculosis protein antigens are known and described in the art and may include, but are not limited to, Mycobacterium tuberculosis major secretory protein antigen 85A (Ag85A), Antigen 85B (Ag85B), Mtb early secretory antigenic target 6 (ESAT-6), Low Molecular Weight Protein Antigen 7 EsxH (Protein TB10.4), and combinations thereof. Protein antigens from other infectious agents are also suitable for use herein. Suitable protein antigens may include bacterial antigens, fungal antigens, viral antigens, parasitic antigens, or antigens from other infectious agents.

The terms “polypeptide,” “peptide,” and “protein,” as used herein, refer to a polymer comprising amino acid residues predominantly bound together by covalent amide bonds. By the term “protein,” we mean to encompass all the above definitions. The terms apply to amino acid polymers in which one or more amino acid residue may be an artificial chemical mimetic of a naturally occurring amino acid, as well as to naturally occurring amino acid polymers and non-naturally occurring amino acid polymers. As used herein, the terms may encompass amino acid chains of any length, including full length proteins, wherein the amino acids are linked by covalent peptide bonds. The protein or peptide may be isolated from a native organism, produced by recombinant techniques, or produced by synthetic production techniques known to one skilled in the art.

In some embodiments, the compositions described herein further comprise a pharmaceutically acceptable carrier. As used herein, “pharmaceutically acceptable carrier” refers to liquid and solid carriers, vehicles, fillers, diluents, encapsulating material, or excipients used in the art for production and delivery of pharmaceutical compositions. Pharmaceutically acceptable carriers are typically non-toxic and inert. A pharmaceutically acceptable carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, pharmaceutically acceptable salts, wetting agents, or other biocompatible materials. A tabulation of ingredients listed by the above categories, may be found in the U.S. Pharmacopeia National Formulary, 1857-1859, (1990).

Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator.

Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like.

A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

The pharmaceutical formulation may additionally include a biologically acceptable buffer to maintain a pH close to neutral (7.0-7.3). Such buffers preferably used are typically phosphates, carboxylates, and bicarbonates. More preferred buffering agents are sodium phosphate, potassium phosphate, sodium citrate, calcium lactate, sodium succinate, sodium glutamate, sodium bicarbonate, and potassium bicarbonate. The buffer may comprise about 0.0001-5% (w/v) of the pharmaceutical formulation, more preferably about 0.001-1% (w/v). Other excipients, if desired, may be included as part of the final pharmaceutical formulation. The remainder of the pharmaceutical formulation may be an acceptable diluent, to 100%, including water. The pharmaceutical formulation may also be formulated as part of a water-in-oil, or oil-in-water emulsion.

In some aspects, provided herein is a method for stimulating an immune cell by contacting the immune cell with the immunostimulatory composition described herein. Suitable immune cells include, for example, antigen presenting cells or T cells. Suitable antigen presenting cells are known in the art and include, for example, dendritic cells, macrophages, Langerhans cells and B cells, among others.

In some embodiments, the immunostimulatory composition is taken up by the immune cell, and the composition is stable within the cell for at least 12 hours, at least 24 hours, at least 2 days, at least 3 days, at least 4 days, at least 5 days, at least 6 days, or at least 7 days.

In some embodiments, the immunostimulatory composition slowly releases the one or more target molecules into the immune cell over at least 12 hours, at least 24 hours, at least 2 days, at least 3 days, at least 4 days, at least 5 days, at least 6 days, at least 7 days, at least 8 days, at least 9 days, or at least 10 days, to stimulate the immune cell.

In some aspects, provided herein is a method for stimulating an immune response to one or more target molecules in a subject in need thereof, the method comprising administering the immunostimulatory composition as described herein in an amount effective to elicit an immune response. In some embodiments, the immune response comprises activation of one or more T cells in the subject. In some embodiments, the T cells are CD1b-restricted.

In another embodiment, the immune response is a humoral immune response. A humoral immune response is mediated by macromolecules found in the extracellular fluids such as secreted antibodies, complement proteins and antimicrobial peptides. Preferably, the humoral immune response is an antibody response to the target molecule. The humoral response preferably includes antibody production specific to the target molecule, and an increase in the effector functions of antibodies, including, for example, pathogen and toxin neutralization, classical complement activation, opsonin promotion of phagocytosis and pathogen elimination, among others.

The term “an effective amount,” as used herein, refers to an amount sufficient to effect a beneficial or desired clinical result. Effective amounts will be affected by various factors that modify the action of the photosensitive nanocarrier upon administration and the subject's biological response to the photosensitive nanocarrier, e.g., the subject's age, sex, and diet, condition to be treated, time of administration, and other clinical factors.

Effective amounts for administration to a human subject can be determined in animal tests and any art-accepted methods for scaling an amount determined to be effective for an animal for human administration. For example, an amount can be initially measured to be effective in an animal model (e.g., to achieve a beneficial or desired clinical result). The amount obtained from the animal model can be used in formulating an effective amount for humans by using conversion factors known in the art. The effective amount obtained in one animal model can also be converted for another animal by using suitable conversion factors such as, for example, body surface area factors.

It is to be understood that, for any particular subject, specific dosage regimes should be adjusted over time according to the individual need and the professional judgment of the person administering or supervising the administration of the photosensitive nanocarrier.

The terms “subject” and “patient” are used interchangeably and refer to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

In some aspects, provided herein is a method for providing a photodynamic therapy to a subject by administering a photosensitive bicontinuous nanosphere nanocarrier described herein and comprising a target molecule to a subject and irradiating the nanocarrier for a time and under conditions sufficient to release the target molecule from the nanocarrier. The irradiation time and conditions are those suitable for, and in some embodiments specific to, the photosensitizer in the nanocarrier. Suitable conditions are described herein.

The term “administration,” as used herein, refers to the introduction of a substance, such as a pharmaceutical formulation, into a subject's body. The administration, e.g., parenteral administration, may include subcutaneous administration, intramuscular administration, transcutaneous administration, intradermal administration, intraperitoneal administration, intraocular administration, intranasal administration and intravenous administration.

The composition according to the invention may be administered to an individual according to methods known in the art. Such methods comprise application, e.g. parenterally, such as through all routes of injection into or through the skin: e.g. intramuscular, intravenous, intraperitoneal, intradermal, mucosal, submucosal, or subcutaneous. Also, the composition may be applied by topical application as a drop, spray, gel or ointment to the mucosal epithelium of the eye, nose, mouth, anus, or vagina, or onto the epidermis of the outer skin at any part of the body.

Other possible routes of application are by spray, aerosol, or powder application through inhalation via the respiratory tract. In this last case, the particle size that is used will determine how deep the particles will penetrate into the respiratory tract.

Alternatively, application may be via the alimentary route, by combining with the food, feed or drinking liquid or water e.g. as a powder, a liquid, or tablet, or by administration directly into the mouth as a: liquid, a gel, a tablet, or a capsule, or to the anus as a suppository.

In some embodiments, the nanocarrier retains a target molecule within a cell for a period of time without loss of cell viability. In some embodiments, the nanocarrier is retained within the cell for at least 12 hours, at least 24 hours, at least 2 days, at least 3 days, at least 4 days, at least 5 days, at least 6 days, or at least 7 days without causing cell death. In some embodiments, the nanocarrier allows the slow release of the target molecule over the time period the nanocarrier is retained within the cell, for example, for at least at least 24 hours, at least 2 days, at least 3 days, at least 4 days, at least 5 days, at least 6 days, or at least 7 days.

In some embodiments, the nanocarrier retains a cytotoxic payload within a cell for a period of time without loss of cell viability. In some embodiments, the nanocarrier is retained within the cell for at least 12 hours, at least 24 hours, at least 2 days, at least 3 days, at least 4 days, at least 5 days, at least 6 days, or at least 7 days without causing cell death.

In some embodiments, when the nanocarrier comprises a photosensitizer, cell death of a cell retaining the photosensitive nanocarrier only occurs following irradiation of the nanocarrier in cases where the nanocarrier.

Unless defined otherwise, all technical and scientific terms used herein have the same meaning as commonly understood by one of ordinary skill in the art to which the invention pertains. All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein, the terms “approximately” or “about” in reference to a number are generally taken to include numbers that fall within a range of 5% in either direction (greater than or less than) the number unless otherwise stated or otherwise evident from the context (except where such number would exceed 100% of a possible value). Where ranges are stated, the endpoints are included within the range unless otherwise stated or otherwise evident from the context.

The present invention has been described in terms of one or more preferred embodiments, and it should be appreciated that many equivalents, alternatives, variations, and modifications, aside from those expressly stated, are possible and within the scope of the invention.

### EXAMPLE 1

The embodiment described here demonstrates the production of bicontinuous nanospheres comprising the photosensitizer pheophorbide A (PhA).

Here, we utilize a highly stable self-assembled polymeric nanocarrier morphology known as a bicontinuous nanosphere (BCN), which is characterized by a cubic lattice of aqueous channels that traverse its hydrophobic interior volume.⁶ BCNs are the polymeric equivalent of lipid cubosomes, and their organized, lyotropic and interconnected internal architecture make them exceptionally robust and stable assemblies, relative to polymersomes, when loading high concentrations of molecular cargo⁷.

BCNs self-assembled from poly(ethylene glycol)-block-poly(propylene sulphide) (PEG-b-PPS) are capable of loading both hydrophilic and hydrophobic compounds simultaneously, making them a versatile drug delivery vehicle.^{7,8} PEG-b-PPS nanocarriers of varying morphology successfully deliver cargo in vitro and in vivo with a demonstrated lack of toxicity in both murine and non-human primate models.^{7,9-13} The hydrophobic PPS block of the polymer can be oxidized into sulfoxide and sulfone derivatives under physiological conditions, leading to an increase in the hydrophilicity of the block and resulting disassembly of PEG-b-PPS nanostructures, including BCN (FIG. 5A).¹⁴ This oxidation-based alteration in the hydrophilic mass fraction of the diblock copolymer drives morphological changes in the aggregate nanostructure, and it has previously been demonstrated that this morphological change can result in the release of cargo from PEG-b-PPS nanostructures.¹² We have previously demonstrated that PEG-b-PPS filomicelles and polymersomes transition into micelles upon photo-oxidation triggered degradation,^{12,14} but how this process impacts intracellular delivery of BCN has not been previously explored.

We hypothesized that PEG-b-PPS BCN nanocarriers would stably retain cargo even upon internalization by cells due to their enhanced nanoarchitecture-dependent stability.⁷ Furthermore, we reasoned that their large hydrophobic PPS volume may serve as a dense sponge for enhanced scavenging of reactive oxygen species (ROS),¹⁵ providing BCN with sensitivity to photo-oxidizers for triggered release but without associated ROS-induced cytotoxicity. To test this, we loaded BCN with the cytotoxic photosensitizer pheophorbide A (PhA) and assessed its ability to safely induce intracellular degradation of BCN. Co-loading of BCN with PhA and cytotoxic chemotherapeutics demonstrated an off-on on-demand photosensitive cytotoxicity (FIG. 5B), verifying that the BCN nanoarchitecture could retain cytotoxic payloads intracellularly with extended cell viability. Such capability may allow usage of more sensitive and a wider range of photosensitizers during photo-triggered intracellular delivery as well as decrease off-target side effects associated with photodynamic nanotherapy.

### Results and Discussion

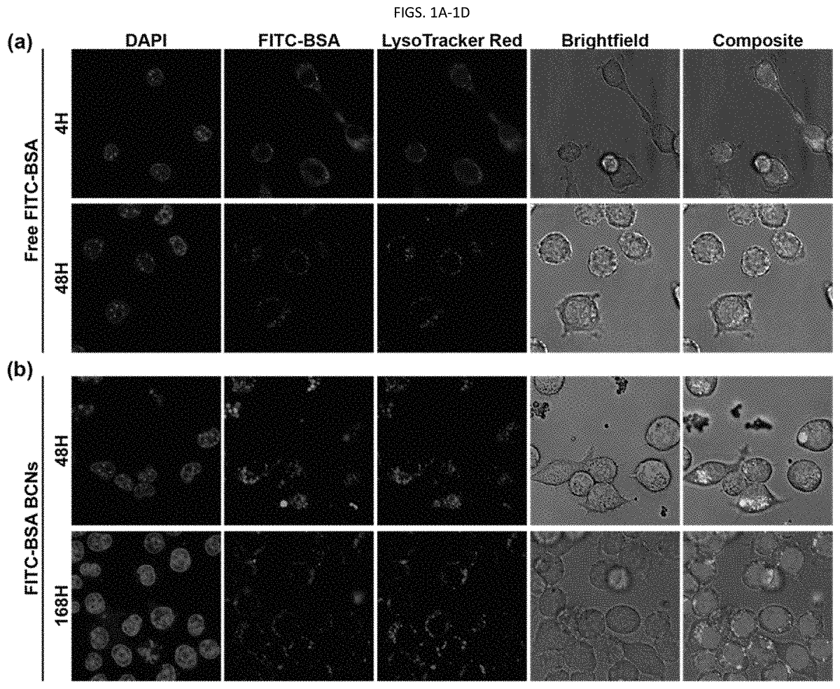

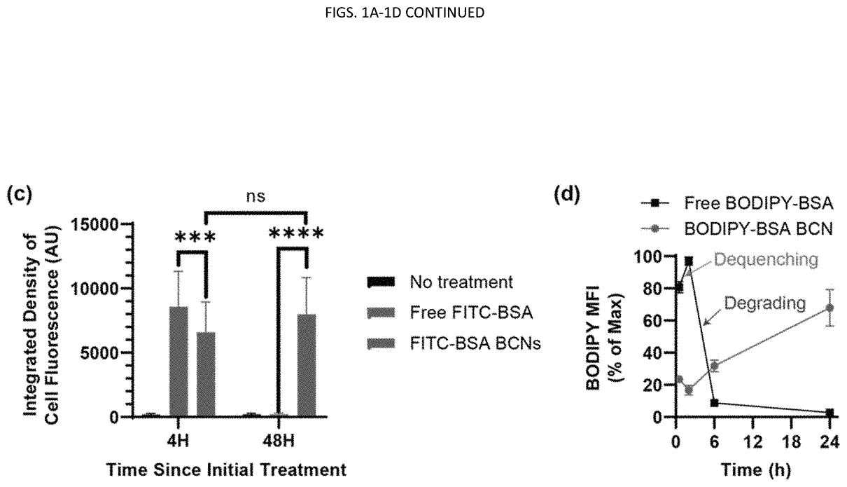

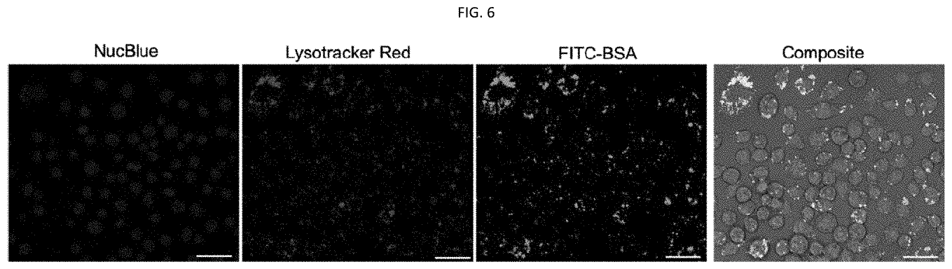

To investigate the ability of BCN to stably retain cargo within cell endosomal compartments, we encapsulated fluorescein-tagged bovine serum albumin (FITC-BSA) within BCNs using flash nanoprecipitation (FNP). FNP is currently the only scalable technique capable of forming uniform spherical BCNs and loading them with hydrophilic and hydrophobic compounds (FIG. 5C).^{8,11,16} Free FITC-BSA was rapidly endocytosed by phagocytic RAW 264.7 cells, forming small punctae that colocalized with a lysosomal stain (FIG. 1A). These FITC-BSA punctae decreased in intensity over the course of 48 h, likely due to pH-dependent degradation of the fluorophore and the BSA, which is known to occur rapidly.^{17,18} In contrast, BCN-encapsulated FITC-BSA retained fluorescence intensity within endosomes (FIG. 6) well beyond 48 h and was still visible within cells 168 h (7 days) after internalization (FIG. 1B). Overall, the intracellular retention at the 168 h timepoint for BCN-loaded FITC-BSA was comparable to free FITC-BSA after 48 h. The persistence of fluorescent punctae confirmed using lysotracker red and lack of FITC-BSA cytosolic signal strongly suggests that BCNs aid in the stable retention of cargo within the lysosomes of cells. Indeed, quantification of fluorescent signal from cells demonstrates that while free FITC-BSA signal was higher at the early 4 h time point compared to FITC-BSA BCNs, it was significantly lower than the FITC-BSA BCN signal at 48 h (FIG. 1C). This precipitous decline in free FITC-BSA intracellular signal is not seen with FITC-BSA encapsulated within BCNs between 4 h and 48 h (FIG. 1C). To further confirm this phenomenon, BCNs were loaded with a BODIPY-BSA and intracellular degradation was quantified over a 24 h period. BODIPY-BSA utilizes a quenched fluorophore, which increases in fluorescence as the BSA is degraded until the BODIPY itself degrades. Free BODIPY-BSA treated cells demonstrated a rapid increase in fluorescence that peaked within 2 h, indicating the nearly immediate degradation of the BSA and resulting dequenching of the BODIPY fluorophores (FIG. 1D). This was followed by a gradual decline in fluorescence as the fluorophore is processed and cleared by the cells. In contrast, BODIPY-BSA encapsulated in BCNs demonstrated a very gradual increase in fluorescence and never peaked over the 24 h period of the study, indicating retarded degradation of the BSA.

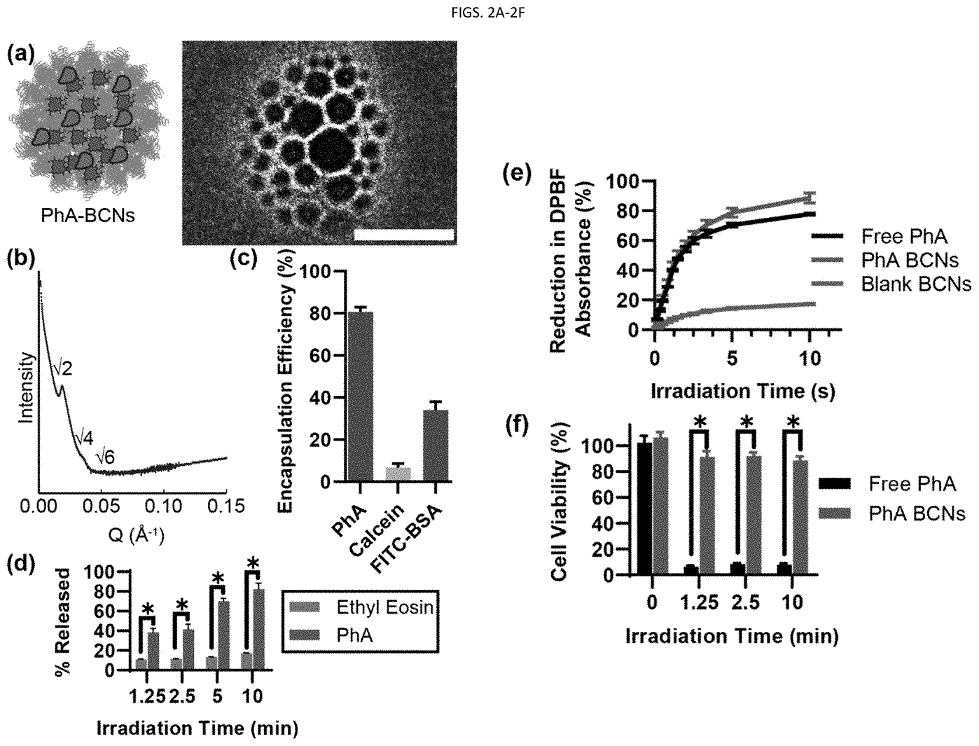

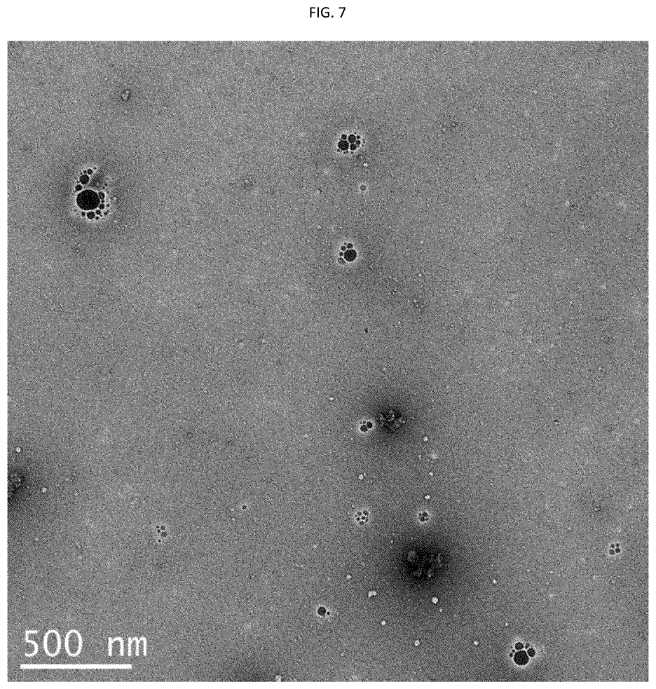

We next sought to probe the stimuli-responsiveness of BCNs by co-encapsulating a photo-oxidizer. We chose pheophorbide A (PhA), known to produce singlet oxygen and ROS at such high levels that it is typically employed to induce cell death for cancer applications.^{19,20} PhA is a relatively hydrophobic molecule (logP=6.93), which correlates well with high encapsulation efficiency in PEG-b-PPS nanostructure systems.^{8,11,13,21} However, encapsulation of hydrophobic cargoes can at times disrupt the aggregate morphology of nanostructures, so we assessed the PhA-loaded BCN (PhA-BCN) formulations for their morphological characteristics.⁷ Negative-stained transmission electron micrographs (TEM) showed that PhA-BCNs retained their characteristic morphology (FIGS. 2A and 7). DLS analysis of the formulations found a diameter of 245.8±11.6 nm and a PDI of 0.15±0.02, which was in good agreement with previous studies on the characteristics of PEG-b-PPS BCNs.^{7,8} Formulations were also analysed by small-angle x-ray scattering for Bragg peaks at the √2, √4, and √6 ratios, indicating that their primitive cubic type (Im3m) internal organization was preserved (FIG. 2B).⁶ As expected for its hydrophobicity, PhA was encapsulated at a high efficiency of 80.7±2.2%. Hydrophilic cargo was simultaneously encapsulated with PhA into BCNs for release studies (FIG. 2C). Agreeing with previously published results, loading of hydrophilic payloads into BCNs is size dependent, with FITC-BSA outperforming calcein encapsulation, 34±4% vs 6.7±1.9%, respectively.⁸

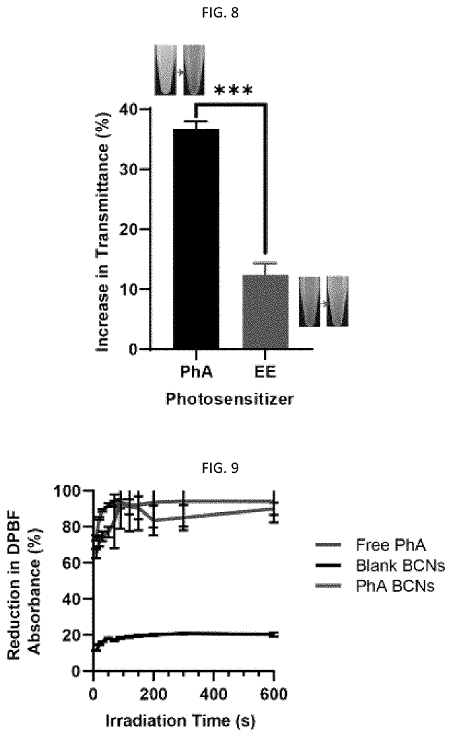

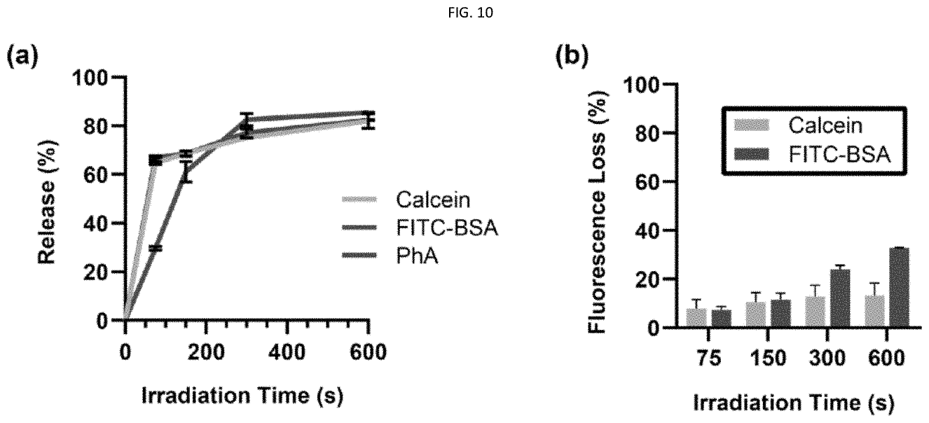

Photo-oxidation of PEG-b-PPS nanostructures had previously been explored using the photo-oxidative fluorophore ethyl eosin.^{8, 14} Other nanostructures, such as polymersomes and filomicelles, appeared to readily release cargo upon photo-oxidation with ethyl eosin, but BCNs contain considerably more PPS in their interior volume and may therefore require a stronger photo-oxidizer. We therefore compared the ability of ethyl eosin and PhA to induce photo-oxidation-triggered degradation of BCNs. After irradiation of a solution of BCNs loaded with photo-oxidizer, the samples were centrifuged to pellet the intact BCNs for sampling of the supernatant. PhA was released into the supernatant in significantly greater amounts than the comparably ineffective ethyl eosin (FIGS. 2D and 8), verifying the superior capacity of PhA to generate the oxidation required for degradation of the dense BCN nanoarchitecture. These results also demonstrated the high stability of BCN and their ability to resist degradation under conditions that would normally rupture polymersomes¹⁴. Upon irradiation, free PhA significantly decreased the absorbance of DPBF, a probe for singlet oxygen generation (FIGS. 2E and 9). Blank BCNs, i.e. BCNs which were not loaded with PhA, showed no effect on DPBF absorbance. Encapsulation of PhA in BCNs did not inhibit the generation of singlet oxygen, with PhA-BCNs demonstrating significantly more singlet oxygen generation than blank BCNs (p<0.0001). This generation of singlet oxygen was able to trigger the release of cargo from BCNs loaded with both PhA and a hydrophilic molecule—either calcein or FITC-BSA. After irradiation, BCNs were centrifuged into a pellet and the supernatant containing released cargo was removed. The pellet was resuspended and assayed for the presence of cargo. Both hydrophilic cargoes were rapidly released from BCNs upon irradiation, most likely due to the destabilization of the BCN morphology allowing for release from the internal aqueous channels (FIG. 10A). This release of cargo, determined by measuring the decrease in fluorescence of the sample, was corrected for any photobleaching effects (FIG. 10B).

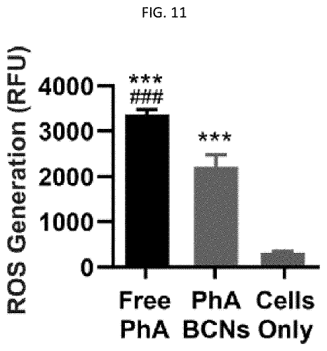

The most common application of PhA is as a photodynamic therapy agent for generating cytotoxic ROS in anti-cancer applications.¹⁹ Indeed, free PhA with very brief irradiation was able to promote significant cell death in RAW 264.7 cells (FIG. 2F). In contrast, PhA-BCNs demonstrated little, though statistically significant (p<0.001), decreases in cell viability after the same duration of irradiation compared to non-irradiated cells, despite forming similar levels of singlet oxygen (FIG. 2E). This difference in ROS generation (FIG. 11) is likely due to the rapid consumption of singlet oxygen species by PPS,¹⁵ which effectively scavenged ROS in the immediate area where they are being formed.

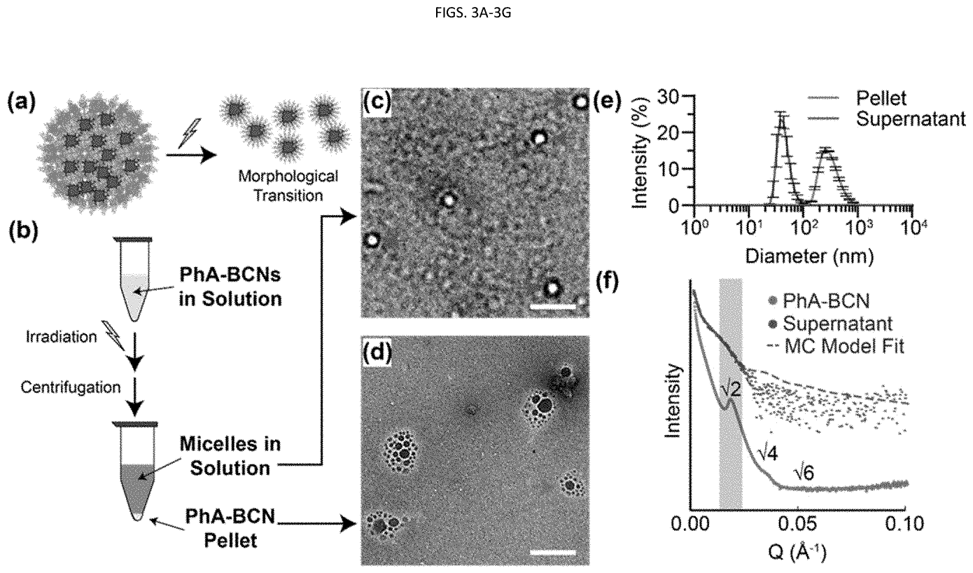

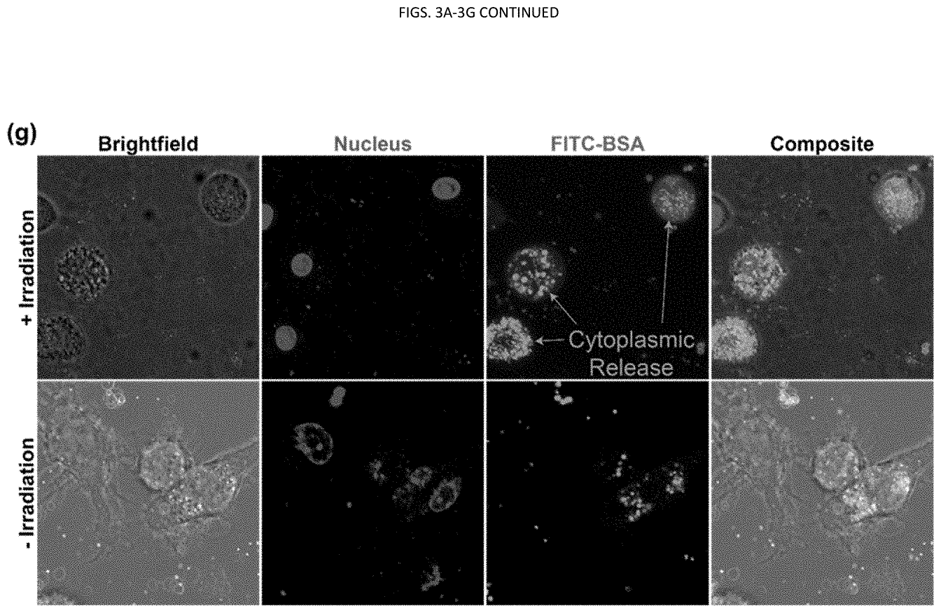

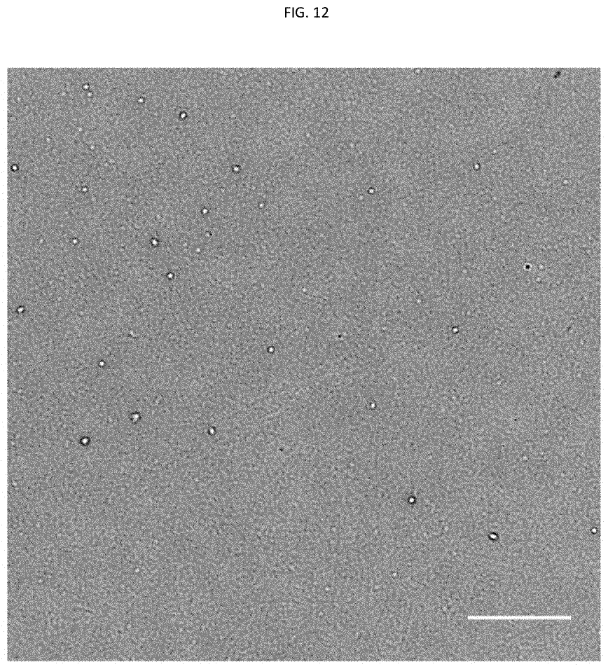

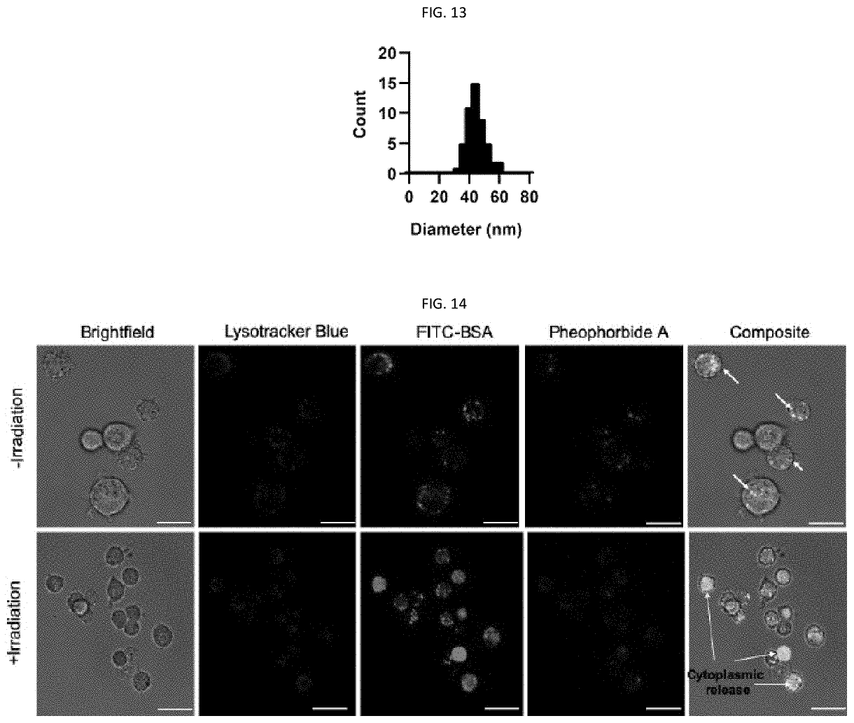

In PEG-b-PPS polymersome and filomicelle systems, photo-oxidation triggers morphological changes, ultimately resulting in the formation of micelle structures.^{12,14} We hypothesized that a similar morphological transition would occur for photo-oxidized BCNs (FIG. 3A). To investigate, we sought to enrich for the morphological transition products by exploiting the propensity of the highly dense BCNs to pellet upon centrifugation (FIG. 3B). Examination of the supernatant and pellet using TEM revealed that the supernatant contained micellar structures while the pellet contained BCNs (FIGS. 3C, 3D and 12). Dynamic light scattering (DLS) size distributions showed a reduction in the diameter of nanostructures after irradiation, representing a shift from larger BCNs to smaller micelles (FIG. 3E), which was also seen after quantification of nanostructure diameters via TEM (FIG. 13). This change in aggregate structure was corroborated by SAXS data, which revealed changes in scattering upon irradiation, particularly the loss of the high intensity crystalline √2 Bragg peak in the supernatant (FIG. 3F). The supernatant SAXS scattering profile was best fit (χ²=0.0005) by a micelle model with core radius of 16.8 nm. Since micellar structures contain a hydrophobic core and are unable to load hydrophilic molecules, this transition from BCN to micelle presents an explanation for the rapid release of hydrophilic cargo after irradiation of PhA-BCNs.

Although often difficult to achieve without cytotoxicity, cytosolic delivery of endosomal payloads is particularly useful for a variety of therapeutic applications, including vaccination.^{9,22} A number of mechanisms have been employed to traverse endosomal membranes, the majority of which induce lysosomal rupture and release of previously sequestered toxic enzymes and ions.²³ In contrast, PEG-b-PPS nanostructures have been found to deliver payloads to the cytosol via temporarily increasing endosomal membrane permeability and without toxicity.^{9,14} For polymersomes, this process involves the oxidation and resulting increased hydrophilicity of the PPS block (FIG. 5A), which triggers a thermodynamically driven conversion from stable vesicles to unstable micelles capable of fusing with and permeabilizing neighbouring membranes for milliseconds.¹⁴ We hypothesized that the morphological transition of BCNs and contemporaneous release of hydrophilic cargo could similarly destabilize endosomal compartments to also allow release of hydrophilic cargo into the cytosol. As a qualitative assessment of this hypothesis, PhA FITC-BSA BCNs, i.e. BCNs co-encapsulating both PhA and FITC-BSA, were incubated with RAW 264.7 cells. Cells were repeatedly washed to remove BCNs that were not internalized and subsequently irradiated prior to confocal imaging. Both irradiated cells and non-irradiated controls contained green fluorescent punctae representing BCNs and cargo localized within the endolysosomal pathway (FIGS. 3G and 14). Non-irradiated cells only featured fluorescence within these punctae, reminiscent of FITC-BSA BCNs imaged in FIGS. 1A-1D. In contrast, irradiated cells featured cytosolic release of the FITC-BSA, visible as a diffuse fluorescence throughout the cytosol.

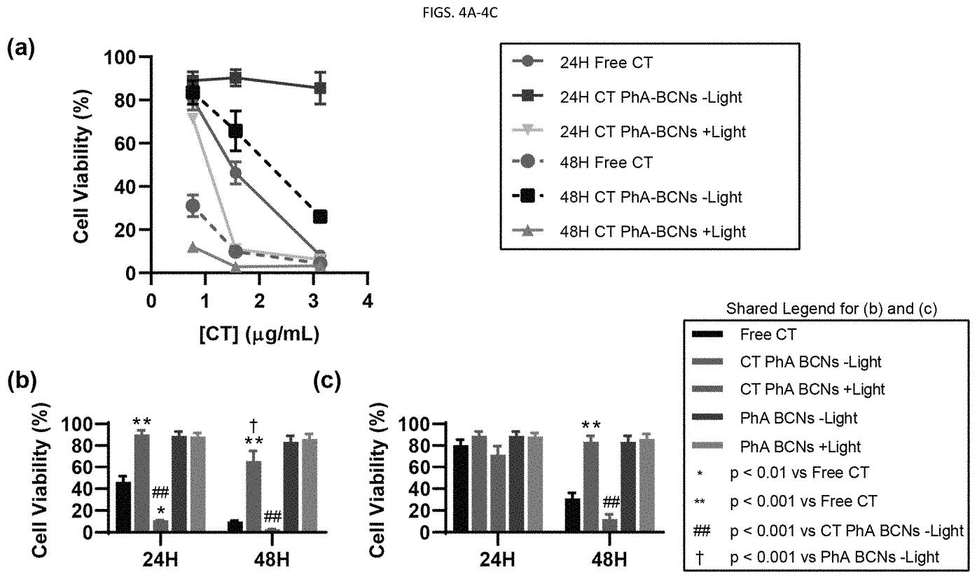

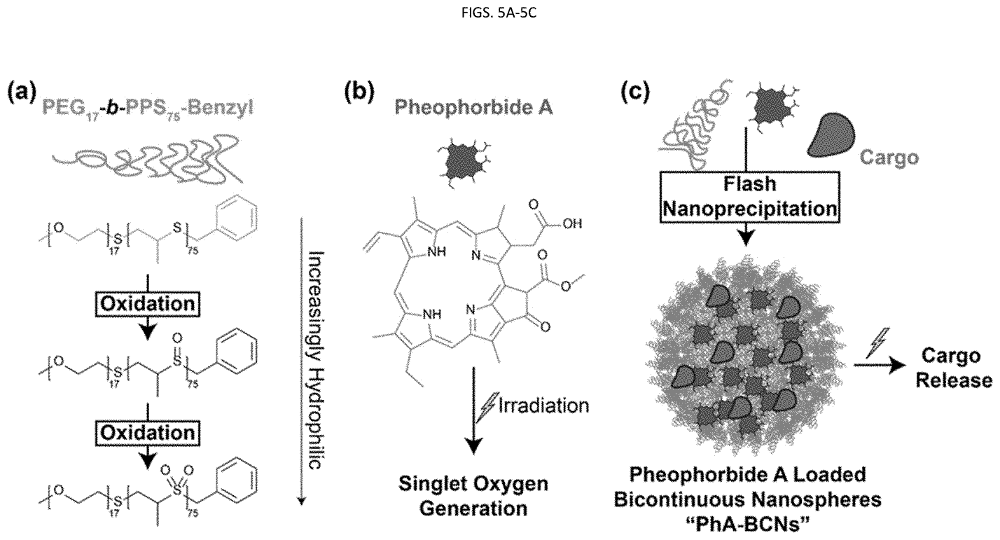

Having demonstrated that PhA-BCNs are both minimally toxic and capable of triggered cytosolic delivery, we sought to assess their ability to protect cells from toxic cargo until on-demand release was triggered for off-on cytotoxicity. Camptothecin is an anti-cancer drug that functions by inhibiting DNA topoisomerase I, a protein present in the nucleus of cells.²⁴ Thus, camptothecin activity is dependent upon cytoplasmic and ultimately nuclear localization, which results in apoptosis and a rapid decrease in cell viability. Reasoning that the release of hydrophilic camptothecin from BCNs would occur upon oxidation of PEG-b-PPS, we loaded BCNs with camptothecin, with or without co-loading of PhA. Camptothecin-loaded PhA-BCNs (CT-PhA-BCNs) containing three concentrations of loaded drug were evaluated for cytotoxicity at 24 and 48 h after an 8 h incubation. Samples were either irradiated or non-irradiated and were also compared to free camptothecin. Non-irradiated CT-PhA-BCNs demonstrated no change in cytotoxicity at 24 h across the three camptothecin concentrations (FIG. 4A). This was statistically very similar to the non-toxic effects of PhA-BCNs as seen in FIG. 2F (p=0.091). Non-irradiated CT-PhA-BCNs did show cytotoxicity at 48 h in a dose dependent manner, suggesting that by that timepoint there is enough release of drug to have a cytotoxic effect at the higher concentrations (FIG. 4A, dagger marker). In contrast to the non-irradiated encapsulated drug, free camptothecin demonstrated dose-dependent cytotoxicity at 24 h and high cytotoxicity regardless of concentration at 48 h. The irradiation of CT-PhA-BCNs, which resulted in cytosolic release of camptothecin, significantly reduced the viability of RAW 264.7 cells. At a camptothecin concentration of 1.56 μg/mL, irradiated CT-PhA-BCNs were significantly more cytotoxic than non-irradiated samples and at 24 h were even significantly more cytotoxic than free drug (FIG. 4B). At the lower 0.63 μg/mL concentration, significant cytotoxic effects were not seen for any treatment at 24 h (FIG. 4C). At 48 h however, both irradiated CT-PhA-BCNs and free CT were significantly more cytotoxic than non-irradiated CT-PhA-BCNs. These results demonstrate that at relevant drug concentrations, BCNs can safely retain drug within endosomal compartments until release is triggered, enhancing both the efficacy and selectivity of on-demand cytotoxicity.

We have demonstrated the first application of photo-sensitive polymeric BCNs by combining the photo-oxidative effects of PhA with oxidation-sensitive PEG-b-PPS. Light-induced oxidation of PEG-b-PPS BCNs resulted in a triggerable in situ morphological transition from bicontinuous to micellar nanostructures. The PEG-b-PPS polymer functioned as an ROS ‘sponge’ to inhibit the typical cytotoxicity and pro-apoptotic effects of PhA. Photo-oxidation of PhA-BCNs within the endosomes of cells resulted in the release of cargo into the cytosol. Such endosomal escape is a critical step in the delivery of many different types of cargo but is difficult to control in an off-on fashion with most triggerable polymeric nanocarriers, as rapid degradation can occur regardless of external stimulation.

Photosensitive self-assembled nanostructures are frequently employed to enhance localized delivery of systemically administered therapeutics. While photo-oxidation will significantly enhance delivery within irradiated tissues, self-assembled nanocarriers will still release payloads within off-target cells due to the degradative environments of the endolysosomal pathway. Here, we find that a bicontinuous nanoarchitecture enhances nanocarrier stability within cells, allowing PEG-b-PPS BCNs to resist degradation and stably retain payloads within cells under conditions that would otherwise rapidly cause disassembly for other self-assembled nanostructures of the same chemical composition, such as PEG-b-PPS polymersomes and filomicelles. We found that BCN protected cells from cytotoxic payloads, including pro-apoptotic photo-oxidizer pheophorbide A while under irradiation as well as the chemotherapeutic camptothecin. Interestingly, photo-oxidation induced a morphological transition of BCNs to a less stable micellar morphology, a process that we characterize via small-angle X-ray scattering and electron microscopy. This morphological transition induced cytosolic delivery of encapsulated camptothecin to both recover and enhance its cytotoxicity. To the best of our knowledge, this is the first demonstration of an inducible bicontinuous-to-micellar transition.

As BCNs inhibited cytoplasmic payload release under normal conditions without irradiation, this highlights the potential use of off-on triggerable photo-oxidation-sensitive platforms for improved control over cytosolic delivery. We demonstrated an advantage of this platform for the intracellular delivery of cytotoxic chemotherapeutics, significantly decreasing apoptosis under ambient conditions while maintain high efficacy upon light stimulation. Such platforms may serve to decrease off-target effects during nanotherapy as well as enhance cell-mediated drug delivery that requires payloads to remain within cells with minimal cytotoxicity, wherein cells can be loaded with therapeutics ex vivo and deliver payloads to specific organs and locations of disease.

### Materials and Methods

Unless specified, all materials were purchased from Sigma-Aldrich (St. Louis, Mo., USA). Pheophorbide A and Camptothecin were obtained from Cayman Chemical (Ann Arbor, Mich., USA). Self-Quenched BODIPY FL Conjugate of BSA (BODIPY-BSA) was acquired from BioVision, Inc (Milpitas, Calif., USA).

Synthesis of PEG-b-PPS Copolymer—PEG₁₇-b-PPS₇₅ was synthesized as described previously.¹⁴ In brief, methoxy-PEG (Mn 750 g/mol) was modified with mesyl chloride to create methoxy-PEG mesylate. The mesylate leaving group was displaced by thioacetic acid to form methoxy-PEG thioacetate. Methoxy-PEG thioacetate (1 eq) was dissolved in 10 mL anhydrous dimethylformamide (DMF) in a schlenk flask under argon. 0.5 M sodium methoxide in methanol (1.1 eq) was added to produce a thiolate anion, and propylene sulfide (75 eq) was added. Ring opening polymerization driven by the thiolate anion as the initiator proceeded for 10 minutes before benzyl bromide (10 eq) was added to end-cap the polymer chains. The reaction proceeded for 4 hours before the DMF was removed by rotary evaporation at 60° C. Crude product was precipitated in methanol to remove impurities. Methanol was decanted, and the product was dried in a vacuum desiccator.

Formation of PEG-b-PPS BCNs—BCNs were formed by flash nanoprecipitation (FNP) technique using a hand-driven CIJ mixer as described previously.^{3, 14} 10 mg of PEG-b-PPS polymer was dissolved in 500 μL of tetrahydrofuran (THF) and was impinged against 500 μL of Milli-Q water. After impingement the nascent BCNs were diluted in a 1.5 mL reservoir of Milli-Q water. THF was removed by vacuum desiccation overnight, resulting in a 5 mg/mL BCN formulation in water. In cases where hydrophobic cargo was loaded into BCNs, the cargo was dissolved along with the 10 mg of PEG-b-PPS polymer in the 500 μL of THF used for impingement. In cases where hydrophilic cargo was loaded into BCNs, the cargo was dissolved in the 500 μL of water used for impingement.

Loading and determination of encapsulation efficiency—The loading of hydrophilic molecules (Calcein, FITC-BSA, BODIPY-BSA) or hydrophobic molecules (Pheophorbide A or Camptothecin) in to BCNs was performed using FNP technique as stated above. Here, 500 μL of Calcien (1 mM) or fluorophore-BSA (2 mg/mL) in water was impinged against 500 μL of Pheophorbide A (0.2 mg/ml) or Camptothecin (0.6 mg/ml) in THF into an aqueous reservoir to form BCNs. After removal of THF via desiccation, BCNs were washed two times with distilled water by centrifugation (10,000×g, 10 min) to remove all the unencapsulated payloads and further resuspended in PBS or water for analysis. The amount of Pheophorbide A was determined by calculating absorbance at 666 nm. Calcein, FITC/BODIPY-BSA, and Camptothecin loading was analyzed by measuring fluorescence intensities at 470/509, 495/525, and 359/434 (excitation/emission, nm), respectively.

Size and Morphological characterization—The size of nanostructures was measured using Dynamic light scattering (DLS) on Zetasizer Nano-ZS (Malvern Instruments, UK). All DLS measurements were performed after 1 in 1000 dilution of samples with PBS.

Transmission electron microscopy (TEM) studies were performed using a 1.0% uranyl formate (UF) in water as negative stain. The stain was adjusted to pH 4.5 by adding 2 μl of 10 N KOH/1 mL of UF. 3 μl of nanoparticle sample (5 mg/mL polymer concentration) was applied to glow discharged carbon-coated copper grids (400-mesh). Samples were passed through two 30 μl volumes of water, and were subsequently negative-stained via passage through two 30 μl volumes of 1% UF. Samples were blotted with Whatman filter paper to remove excess stain. Roughly 0.5 μl stain remains on the grid following this procedure with an activity of 2.55×10⁵ μCi/grid. Images were acquired at 30,000× on a JOEL 1400 Transmission Electron Microscope operating at 120 kV.

Small angle X-ray scattering (SAXS) experiments were performed at the DuPont-Northwestern-Dow Collaborative Access Team (DND-CAT) beamline at Argonne National Laboratory's Advanced Photon Source (Argonne, Ill., USA) with 10 keV (wavelength λ=1.24 Å) collimated X-rays. All the samples were measured in the q-range 0.001 to 0.5 Å−¹ and silver behenate was utilized to calibrate the q-range. The data reduction was made using PRIMUS 2.8.2 software, where the final scattering curve was obtained after subtraction of solvent buffer scattering. The micelle model fitting was performed using SasView.¹⁵

In vitro Singlet oxygen generation studies—In vitro singlet oxygen generation was determined by chemical oxidation of 1,3-Diphenylisobenzofuran (DPBF). A stock solution (2 mM) of DPBF was prepared in dimethyl sulfoxide and then diluted to 100 μM using water for further analysis. A 1:1 ratio of DPBF and samples (6 μg/mL of free PhA or 6 μg/mL PhA loaded BCNs or Blank BCNs) were prepared. A 200 μL of this mixture was transferred in to a 96 well black plate and then irradiated immediately using Max-303 Xenon Light Source (385 to 740 nm, Asahi Spectra) at different light intensities (5 and 50 mW/cm²) for 10 min. At predetermined time intervals DPBF absorbance was measured at 415 nm and singlet oxygen generation was then calculated as percentage reduction in DPBF absorbance as compared to the initial DPBF absorbance.

In vitro BCN degradation and Release of payloads—In vitro degradation studies of PhA BCNs were performed under irradiation. Briefly, 200 μL of PhA BCNs (1 mg/ml) was transferred in to multiple wells of a 96 well black plate and then irradiated for 2.5 min at 50 mW/cm². The irradiated samples were immediately collected and centrifuged at 10,000×g for 10 min. After centrifugation, the supernatants and pellets were collected separately and analyzed using DLS and TEM as described above.

In vitro release of calcein, FITC-BSA, ethyl eosin and PhA from BCNs was measured after irradiation. 200 μL of PBS containing PhA BCNs co-loaded with calcein or FITC-BSA, or PhA BCNs and ethyl eosin BCNs, were transferred in to multiple wells of a 96 well black plate and then irradiated at 50 mW/cm² for 1.25, 2.5, 5 and 10 minutes. Post irradiation samples were centrifuged at 10,000×g for 10 min and supernatants were collected and further analyzed for amount released as described earlier. For PhA BCNs and ethyl eosin BCNs, % transmittance was measured using an M3 plate reader.

Cell culture—RAW 264.7 cells (murine macrophage cell line) were acquired from American Type Culture Collection (ATCC, Rockville, Md., USA) were employed for cell culture experiments. Cells were cultured in Dulbecco's Modified Eagle's Medium (DMEM) supplemented with 10% fetal bovine serum (FBS), penicillin (100 IU/mL) and streptomycin (100 μg/mL) at 37° C. in the presence of air (95%) and CO₂ (5%).

Cellular uptake studies—RAW 264.7 cells (2.5×10⁵ cells/mL, 500 μL) were seeded in each well of a 48-well plate and adhered overnight. The adhered cells were added with 50 μL of free BODIPY-BSA (200 μg/mL) or BODIPY-BSA BCNs (PBS) and incubated for 4 h. The polymer and BODIPY-BSA concentration in each well were calculated as 0.5 mg/mL and 20 μg/mL, respectively. After incubation, cells were washed two times with PBS, collected in to flow cytometry tubes and incubated with 50 μEL Zombie Aqua (1:100) fixable cell viability dye (Biolegend, San Diego, Calif.) for 15 min at 4° C. Then the cells were washed with 500 μL PBS, resuspended in cell staining buffer and analyzed using a BD Fortessa flow cytometer. The cellular uptake was measured as median fluorescence intensity (MFI) in the FITC channel, demonstrating the amount of BODIPY-BSA taken up by each cell.

MTT assay—RAW 264.7 cells (2×10⁵ cells/mL, 100 μL) were plated in each well of a black 96-well plate and adhered overnight. The adhered cells were treated with free PhA (DMSO: PBS, 1:3) or PhA loaded BCNs (PBS) or CT BCNs or PhA-CT BCNs and incubated for 4 h. After incubation, cells were immediately irradiated with at a light intensity of 50 mW/cm² for 1.25 and 2.5 minutes. Post irradiation, cells were incubated for predetermined time intervals and then added with MTT (5 mg/mL in PBS, 10 μL). After 4 h MTT incubation with cells, formazan crystal deposition in each well was dissolved in DMSO (200 μL) and the absorbance was measured at 560 nm. All the samples were analyzed in quadruplicates.

The percentage cell viability was calculated as:

% cell viability=(OD of treated sample/OD of untreated sample)*100.

Intracellular ROS generation—2′,7′-dichlorofluorescin diacetate (DCFDA) assay was utilized to measure intracellular ROS generation. Briefly, RAW 264.7 cells (2×10⁵ cells/mL, 100 μL) were seeded in each well of a black 96-well plate and adhered overnight. The adhered cells were incubated with free PhA (DMSO: PBS, 1:3) or PhA loaded BCNs (PBS) or left untreated. After 4 h incubation, cells were washed two times with PBS and incubated with DCFDA (10 μM) for 45 min in the dark. Cells were then rinsed with PBS, irradiated (50 mW/cm², 2.5 min) and fluorescence was measured (ex/em, 485/535 nm) immediately using a microplate reader.

Confocal microscopy—RAW 264.7 cells (1×10⁵ cells/mL, 300 μL) were seeded in each well of an 8-well Chamber slide (Thermo Fischer Scientific) and adhered overnight. The adhered cells were incubated with FITC-BSA loaded BCNs or PhA co-loaded with FITC-BSA BCNs for 4 h. After incubation, cells were washed two times with PBS and then added with 300 μL DMEM. Then the 8-well slides were irradiated at a light intensity of 50 mW/cm² for 2.5 min or kept under dark conditions. Post irradiated cells was then washed with PBS, added with 300 μL PBS and incubated with NucBlue™ Live ReadyProbes™ Reagent (nuclei stain, 1 drop) for 15 min in the dark. A similar procedure was followed for control slides that were kept under dark conditions. Wherever required, Invitrogen™ Lysotracker (Lysotracker Red™ DND-99 or Lysotracker Blue™ DND-22, 1 in 2000 dilution in DMEM) was incubated with cells for 45 min to stain lysosomes. The 8-well slides were then imaged within a humidified chamber using a 63× oil-immersion objective on a SP5 Leica Confocal Microscope using HyD detectors and lasers. Data analysis was performed using ImageJ Software.

### REFERENCES

1 Petschauer, J. S., Madden, A. J., Kirschbrown, W. P., Song, G. & Zamboni, W. C. The effects of nanoparticle drug loading on the pharmacokinetics of anticancer agents. Nanomedicine (Lond) 10, 447-463, doi:10.2217/nnm.14.179 (2015).

2 Wang, A. Z., Langer, R. & Farokhzad, O. C. Nanoparticle delivery of cancer drugs. Annu Rev Med 63, 185-198, doi:10.1146/annurev-med-040210-162544 (2012).

3 Zamboni, W. C. et al. Best practices in cancer nanotechnology: perspective from NCI nanotechnology alliance. Clin Cancer Res 18, 3229-3241, doi:10.1158/1078-0432.CCR-11-2938 (2012).

4 Mura, S., Nicolas, J. & Couvreur, P. Stimuli-responsive nanocarriers for drug delivery. Nat Mater 12, 991-1003, doi:10.1038/nmat3776 (2013).

5 Scarpa, E. et al. Quantification of intracellular payload release from polymersome nanoparticles. Scientific Reports 6, 29460, doi:10.1038/srep29460 (2016).

6 Allen, S. D., Bobbala, S., Karabin, N. B. & Scott, E. A. On the advancement of polymeric bicontinuous nanospheres toward biomedical applications. Nanoscale Horiz 4, 258-272, doi:10.1039/c8nh00300a (2019).

7 Allen, S. D., Bobbala, S., Karabin, N. B., Modak, M. & Scott, E. A. Benchmarking Bicontinuous Nanospheres against Polymersomes for in Vivo Biodistribution and Dual Intracellular Delivery of Lipophilic and Water-Soluble Payloads. ACS Applied Materials & Interfaces 10, 33857-33866, doi:10.1021/acsami.8b09906 (2018).

8 Bobbala, S., Allen, S. D. & Scott, E. A. Flash nanoprecipitation permits versatile assembly and loading of polymeric bicontinuous cubic nanospheres. Nanoscale 10, 5078-5088, doi:10.1039/c7nr06779h (2018).

9 Scott, E. A. et al. Dendritic cell activation and T cell priming with adjuvant- and antigen-loaded oxidation-sensitive polymersomes. Biomaterials 33, 6211-6219, doi:10.1016/j.biomaterials.2012.04.060 (2012).

10 Yi, S. et al. Tailoring Nanostructure Morphology for Enhanced Targeting of Dendritic Cells in Atherosclerosis. ACS Nano 10, 11290-11303, doi:10.1021/acsnano.6b06451 (2016).

11 Allen, S., Osorio, O., Liu, Y. G. & Scott, E. Facile assembly and loading of theranostic polymersomes via multi-impingement flash nanoprecipitation. J Control Release 262, 91-103, doi:10.1016/j.jconrel.2017.07.026 (2017).

12 Karabin, N. B. et al. Sustained micellar delivery via inducible transitions in nanostructure morphology. Nat Commun 9, 624, doi:10.1038/s41467-018-03001-9 (2018).

13 Allen, S. D. et al. Celastrol-loaded PEG-b-PPS nanocarriers as an anti-inflammatory treatment for atherosclerosis. Biomater Sci-Uk 7, 657-668, doi:10.1039/c8bm01224e (2019).

14 Vasdekis, A. E., Scott, E. A., O'Neil, C. P., Psaltis, D. & Hubbell, J. A. Precision intracellular delivery based on optofluidic polymersome rupture. ACS Nano 6, 7850-7857, doi:10.1021/nn302122h (2012).

15 Rajkovic, O. et al. Reactive Oxygen Species-Responsive Nanoparticles for the Treatment of Ischemic Stroke. Advanced Therapeutics 2, 1900038, doi:10.1002/adtp.201900038 (2019).

16 Allen, S., Vincent, M. & Scott, E. Rapid, Scalable Assembly and Loading of Bioactive Proteins and Immunostimulants into Diverse Synthetic Nanocarriers Via Flash Nanoprecipitation. J Vis Exp, doi:10.3791/57793 (2018).

17 Humphries, W. H. t. & Payne, C. K. Imaging lysosomal enzyme activity in live cells using self-quenched substrates. Analytical biochemistry 424, 178-183, doi:10.1016/j.ab.2012.02.033 (2012).

18 Midoux, P., Roche, A. C. & Monsigny, M. Quantitation of the binding, uptake, and degradation of fluoresceinylated neoglycoproteins by flow cytometry. Cytometry 8, 327-334, doi:10.1002/cyto.990080314 (1987).

19 Busch, T. M., Cengel, K. A. & Finlay, J. C. Pheophorbide a as a photosensitizer in photodynamic therapy In vivo considerations. Cancer Biol Ther 8, 540-542, doi:DOI 10.4161/cbt.8.6.8067 (2009).

20 Tang, P. M., Liu, X. Z., Zhang, D. M., Fong, W. P. & Fung, K. P. Pheophorbide a based photodynamic therapy induces apoptosis via mitochondrial-mediated pathway in human uterine carcinosarcoma. Cancer Biol Ther 8, 533-539, doi:10.4161/cbt.8.6.7694 (2009).

21 Yi, S. J. et al. Surface Engineered Polymersomes for Enhanced Modulation of Dendritic Cells During Cardiovascular Immunotherapy. Adv Funct Mater, doi:ARTN 1904399 10.1002/adfm.201904399 (2019).

22 Shete, H. K., Prabhu, R. H. & Patravale, V. B. Endosomal escape: a bottleneck in intracellular delivery. J Nanosci Nanotechnol 14, 460-474, doi:10.1166/jnn.2014.9082 (2014).

23 Varkouhi, A. K., Scholte, M., Storm, G. & Haisma, H. J. Endosomal escape pathways for delivery of biologicals. J Control Release 151, 220-228, doi:10.1016/j.jconrel.2010.11.004 (2011).

24 Liu, L. F. et al. Mechanism of action of camptothecin. Ann Ny Acad Sci 922, 1-10 (2000).

### EXAMPLE 2

The embodiment described here demonstrates that an intracellular bicontinuous nanosphere (BCN) containing immunostimulants can retain the immunostimulants and slowly releases them to elicit an immune response, therefore useful for the purposes of vaccination by allowing a sustained and superior immune response following immunization.

The following data shows how a unique subset of T cells can be stimulated to proliferate at longer time points following immunization with MA-loaded BCNs compared to a standard nanoparticle composed of poly(lactic-co-glycolic acid) (PLGA).

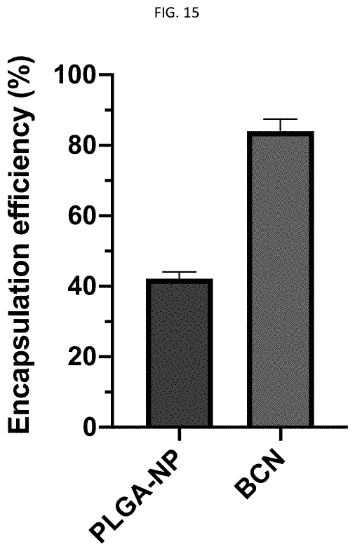

We have made bicontinuous nanospheres containing mycolic acid (MA) using the methods described herein. MA is a major lipid component of the mycobacterial cell wall and is under investigation as a component of vaccine formulations. Delivery of MA to the endosomes of certain human antigen presenting cells can result in activation of CD1b-restricted T cells, and thus the mechanism by which nanoparticles deliver MA is important for the therapeutic effect. Compared to poly(lactic-co-glycolic acid)-based nanoparticles (PLGA NP), BCNs have higher MA encapsulation efficiency. As shown in FIG. 15, encapsulation efficiency of MA in BCN is 95+/−3%, whereas PLGA NP only has an encapsulation efficiency of MA of 43+/−2%. Encapsulation was calculated using BCNs or PLGA NP loaded with MA functionalized with 4-bromomethyl-6,7-dimethoxycoumarin. The fluorescence of these particles were then measured using a spectrophotometer (λex=365 nm, λem=410 nm). PLGA is used as a control here as it is an FDA approved copolymer that is considered a standard in the field of nanoparticle-based drug delivery.

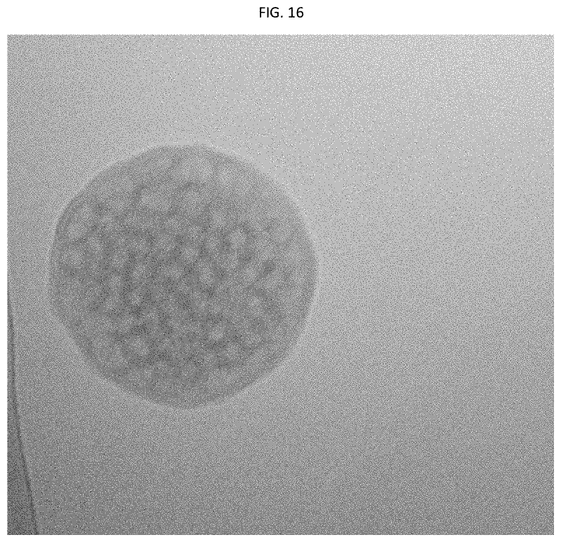

As shown in the Cryo-TEM image of a BCN loaded with MA in FIG. 16, no structural modulation was observed in BCNs loaded with MA.

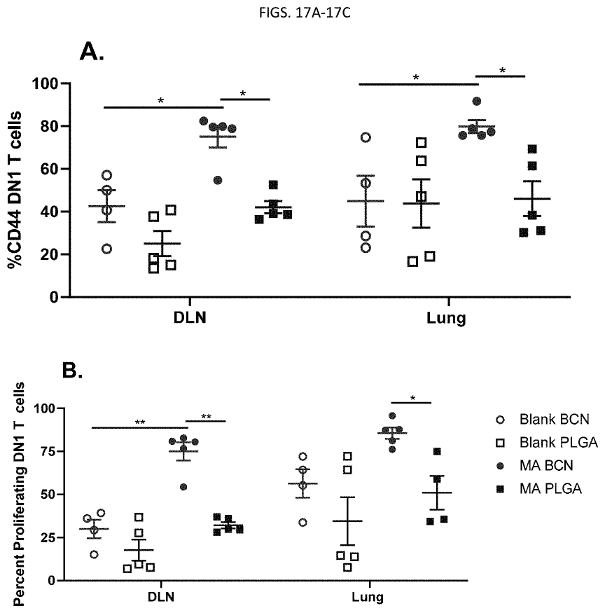

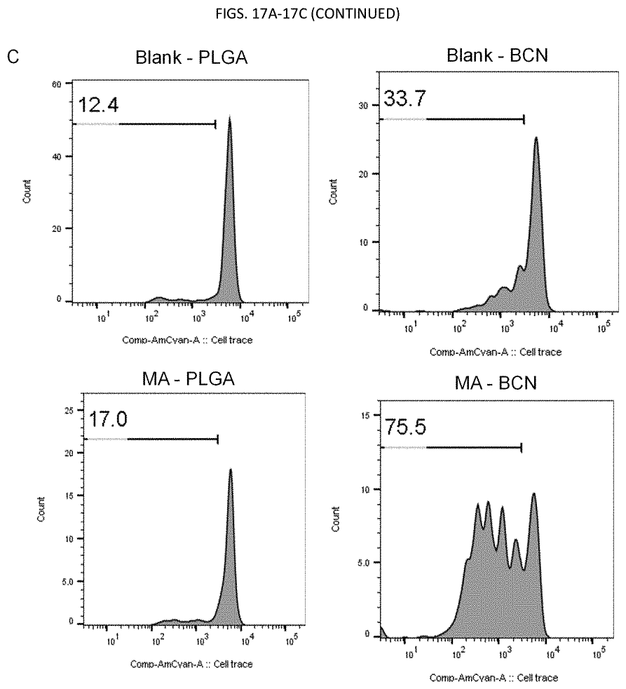

Compared to unloaded BCNs, MA-loaded PLGAs, and unloaded PLGAs, vaccination with MA-loaded BCNs allows for higher activation and proliferation. As demonstrated in FIGS. 17A and 17B, the percentage of CD44+ DN1 T cells (FIG. 17A) or proliferating DN1 T cells (FIG. 17B) (CD1b-restricted T cells from humanized mice) taken from draining lymph nodes (DLNs) and lungs of hCD1Tg mice seven days post 3e6 intravenous DN1 T cell adoptive transfer and 6 days after intratracheal vaccination by BCNs loaded with MA is higher than unloaded BCNs or PLGAs and PLGAs loaded with MA. In addition, FIG. 17C demonstrates that DN1 T cell proliferation of hCD1Tg DLN, 7 days post 3e6 IV DN1 T cell adoptive transfer and 6 days after intratracheal vaccination as shown with cell trace BV 541 by BCNs loaded with MA is higher than unloaded PLGAs or BCNs and MA loaded PLGA.

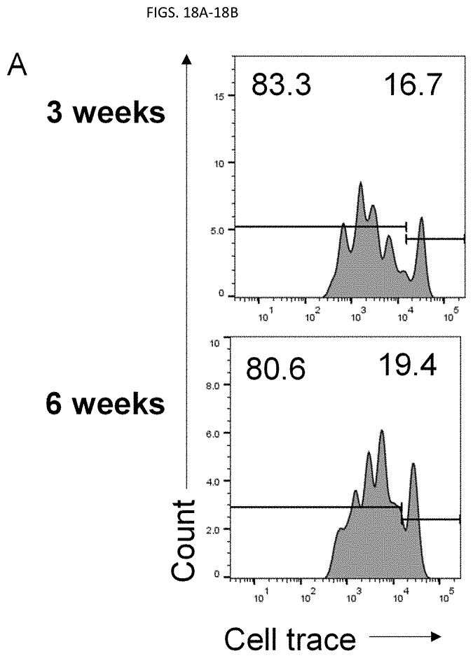

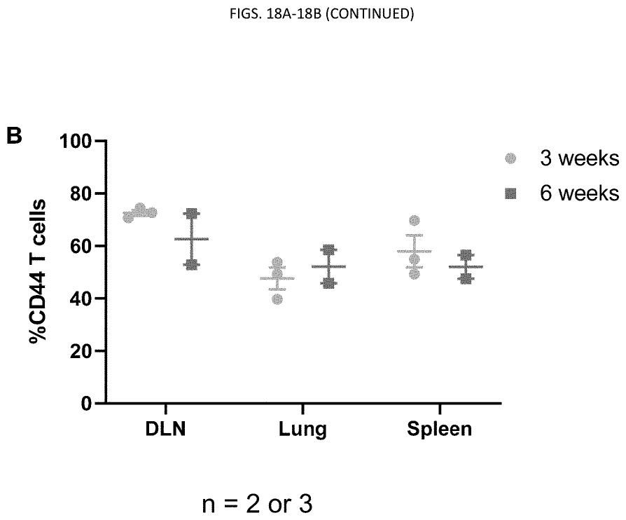

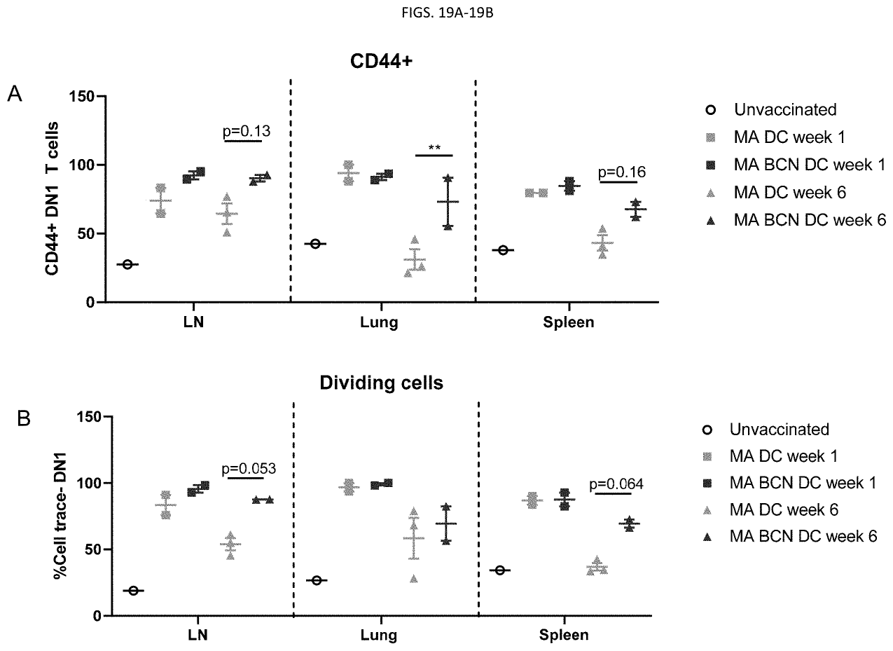

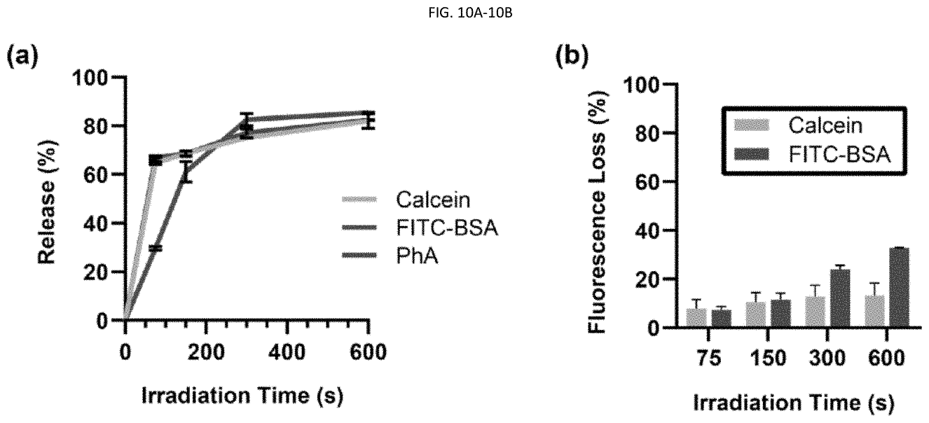

We also discovered that adoptively transferred DN1 T cells are activated and proliferate 3 and 6 weeks after vaccination by MA-loaded BCNs in hCD1Tg mice (humanized transgenic mice that respond to lipid antigens similar to humans). CD44+ DN1 T cells were obtained from draining lymph nodes (DLN), lung and spleen 3 or 6 weeks after intratracheal vaccination of MA-loaded BCNs in hCD1Tg mice. Mice were adoptively transferred with 3e6 DN1 T cells 1 week prior to the 3 and 6 week timepoints. FIG. 18A shows the cell trace of CD44+ DN1 T cells after 3 or 6 weeks. FIG. 18B displays the percentage of CD44+ DN1 T cells in DLN, lung, or spleen 3 or 6 weeks after intratracheal vaccination of MA-loaded BCNs.

We also conducted experiments to show that MA BCN and MA pulsed BMDC (bone marrow-derived dendritic cells) vaccination displays differences in activation and proliferation of DN1 T cells 6 weeks after vaccination, but not 1 week after vaccination. hCD1Tg BMDC were treated ex vivo with 10 μg/mL MA or with 5 μg/mL MA BCN for 18 h. hCD1Tg mice were then intratracheally vaccinated with these cells at 1 or 6 weeks before sacrifice. One week before the 1 and 6 week timepoints, 3e6 DN1 T cells were adoptively transferred intravenously. DLN, lung, and spleen were obtained. The percentage of CD44+ DN1 T cells after vaccination displayed in FIG. 19A shows activation. The percentage of cell trace-DN1 T cells after vaccination displayed in FIG. 19B shows proliferation.

We claim:

1. An intracellularly stable nanocarrier comprising a bicontinuous nanosphere nanocarrier comprising poly(ethylene glycol)-block-poly(propylene sulphide) (PEG-b-PPS), mycolic acid (MA) and one or more target molecules, wherein the intracellularly stable nanostructures is retained within a target cell and can slow release its contents over time.

2. The intracellularly stable nanocarrier of claim 1, wherein the nanocarrier is capable of slow-releasing the target molecule over 4-7 days.

3. An immunostimulatory composition comprising the intracellular stable nanocarrier of claim 1, wherein the immunostimulatory composition is capable of stimulating an immune response to the target molecule.

4. The immunostimulatory composition of claim 3, wherein the one or more target molecules is an antigen.

5. The immunostimulatory composition of claim 4, wherein the antigen is a protein antigen.

6. The immunostimulatory composition of claim 3, wherein the one or more target molecules is hydrophobic or hydrophilic

7. The immunostimulatory composition of claim 3, wherein the one or more target molecules is selected from the group consisting of a DNA molecule, an RNA molecule, a plasmid, a peptide, a protein, a small molecule, and combinations thereof.

8. The immunostimulatory composition of claim 3, wherein the composition further comprises a photosensitizer.

9. The immunostimulatory composition of claim 8, wherein the photosensitizer is hydrophobic and incorporates into an interior hydrophobic volume of the bicontinuous nanosphere.

10. The immunostimulatory composition of claim 8, wherein the photosensitizer is pheophorbide A (PhA).

11. The immunostimulatory composition of claim 3, wherein the composition is stable within a cell for at least 4 days, preferably at least 5 days.

12. A method of stimulating an immune cell, the method comprising contacting the immune cell with the immunostimulatory composition of claim 3, wherein the immunostimulatory composition stimulates the immune cell.

13. The method of claim 12, wherein the immunostimulatory composition is taken up by the immune cell, and wherein the composition is stable within the cell for at least 4-7 days.

14. The method of claim 12, wherein the immunostimulatory composition slowly releases the one or more target molecules into the immune cell over at least seven days to stimulate the immune cell.

15. The method of claim 12, wherein the immune cell is in vivo within a subject.

16. A method of stimulating an immune response to one or more target molecules in a subject in need thereof, the method comprising administering the immunostimulatory composition of claim 3 in an amount effective to elicit an immune response.

17. The method of claim 16, wherein the immune response comprises activation of one or more T cells in the subject.

18. The method of claim 17, wherein the T cells are CD1b-restricted.

19. The immunostimulatory compound of claim 3, wherein the one or more target molecule is cytotoxic.

20. The immunostimulatory composition of claim 3, wherein the PEG-b-PPS is PEG₁₇-b-PPS₇₅.

21. The immunostimulatory composition of claim 3, wherein the PEG-b-PPS is benzyl functionalized.

22. The immunostimulatory composition of claim 3, further comprising a pharmaceutically acceptable carrier.

23. A method for providing a photodynamic therapy to a subject comprising:
(i) administering to the subject the immunostimulatory composition of claim 8;
(ii) irradiating the composition comprising the nanocarrier for a time and under conditions sufficient to generate reactive oxygen species and release the one or more target molecules from the nanocarrier.

24. The method of claim 23, wherein the nanocarrier is irradiated with light at an intensity between 5 and 200 mW/cm² for about 0.5 minutes to about 5 minutes.

25. The method of claim 24, wherein the light has a wavelength of 385 nm to 740 nm.

26. The method of claim 23, wherein the one or more target molecules is cytotoxic.
